(19) United States
(12) Patent Application Publication
(10) Pub. No.: US 2010/0196986 A1
(43) Pub. Date: Aug. 5, 2010

(54) COMPOSITION

(75) Inventors: Brian Sogaard Laursen, Solbjerg (DK); Jakob Broberg Kristensen, Langa (DK)

Correspondence Address: STEPTOE & JOHNSON LLP, 1330 CONNECTICUT AVENUE, N.W., WASHINGTON, DC, 20036, US

(73) Assignee: Danisco A/S, Copenhagen K (DK)

(21) Appl. No.: 12/700,902

(22) Filed: Feb. 5, 2010

Related U.S. Application Data
(63) Continuation of application No. PCT/IB2009/006255, filed on Jun. 24, 2009.
(60) Provisional application No. 61/150,127, filed on Feb. 5, 2009.
(60) Provisional application No. 61/099,639, filed on Sep. 24, 2008.

(30) Foreign Application Priority Data
Jun. 25, 2008 (GB) 0811661.8
Feb. 5, 2009 (GB) 0901965.4

(51) Int. Cl. C12N 9/04; C12N 9/00; C12N 9/02

(52) U.S. Cl. 435/190; 435/183; 435/189

(57) ABSTRACT

There is provided an anti-fouling composition comprising
(i) a surface coating material;
(ii) (a) a first enzyme; and
   (b) a first substrate, wherein action of the first enzyme on the first substrate provides a second substrate
(iil) a second enzyme, wherein the second enzyme is encapsulated in a silicate, and wherein said second enzyme generates an anti-foulant compound when acting on said second substrate.

The present invention relates to an anti-fouling composition. In particular, the present invention relates to an anti-fouling composition comprising an enzyme capable of producing a compound having an anti-fouling effect.

Biofouling is a problem at any surface that is constantly or intermittently in contact with water. Attachment and growth of living organisms on surfaces causes hygienic and functional problems to many types of equipment and devices ranging from medical to implants and electronic circuitry to larger constructions, such as processing equipment, paper mills and ships.

In many cases, biofouling consists of microscopic organic impurities or a visible slimy layer of extracellular polymeric substances (EPS) containing bacteria and other microorganisms. This category of biofouling is called microfouling, or more commonly biofilm, and occurs everywhere in both natural and industrial environments where surfaces are exposed to water. When fully developed, biofouling in marine environments also includes macroscopic organisms, such as algae and barnacles. This type of biofouling is a particular problem for submerged structures, such as pipelines, cables, fishing nets, the pillars of bridges and oil platforms and other port or hydrotechnical constructions. Fuel consumption of ships may be increased by up to 40% due to biofouling.

In particular, as discussed in U.S. Pat. No. 5,071,479, the growth of marine organisms on the submerged parts of a ship's hull is a particular problem. Such growth increases the frictional resistance of the hull to passage through water, leading to increased fuel consumption and/or a reduction in the speed of the ship. Marine growths accumulate so rapidly that the remedy of cleaning and repainting as required in dry-dock is generally considered too expensive.

Coatings specialised for inhibiting fouling are mainly of two different types, namely foul-release and self-polishing paints. Foul-release paints are characterised by a smooth surface which makes settlement difficult for fouling organisms. However, this mechanism is generally only effective for ships that spend most of the time sailing and preferentially at high cruising speeds. As the name indicates, the self-polishing paints are gradually dissolved or polished away. This provides continuous access to a fresh layer of paint. These paints may contain antifouling agents that are gradually released from the coating at a concentration which is high enough to inhibit fouling by marine organisms at the hull surface.

Previously, tributyl tin (TBT) has been a widely used biocide, particularly in marine anti-fouls. However, due to growing concerns about the environmental effects caused by using such organic tin biocides at their commercial levels as an antifoulant active ingredient in coating compositions for aquatic (marine) applications the use has effectively been stopped. It has been shown that, due to the widespread use of to tributyltin-type compounds in particular, at concentrations as high as 20 wt. % in paints for ship bottoms, the pollution of surrounding water due to leaching has reached such a level as to cause the reproductive and immune defects in mussel and shell organisms. These effects have been detected along the French British coastline and a similar effect has been confirmed in U.S. and Far East waters. The International Maritime Organisation (IMO) International Convention on the Control of Harmful Anti-Fouling Systems (AFS Convention) adopted at an IMO diplomatic conference in October 2001 banned application of TBT coatings on ships with effect from 1 Jan. 2003 followed, as of 1 Jan. 2008, by the elimination of active TBT coatings from ships.

Currently, the most widely used antifouling paints are based on copper with booster biocides (Yebra et al, 2004. Progress in Organic Coatings 50:75-104). Booster biocides e.g. copper pyrithione or isothiazolone are however necessary to complement the biocidel action of copper, which is ineffective against some widespread algal species tolerant to copper (e.g. Enteromorpha spp). The booster biocides are equally under suspicion for being harmful to the environment. The safety of booster biocides has been reviewed by several authors (Boxall, 2004. Chemistry Today 22(6):46-8; Karlsson and Eklund, 2004. Marine Pollution Bulletin 2004; 49:456-64; Kobayashi and Okamura, 2002. Marine Pollution Bulletin 2002; 44:748-51; Konstantinou and Albanis, 2004. Environment International 2004; 30:235-48; Ranke and Jastorff, 2002 Fresenius Environmental Bulletin 2002; 11(10a):769-72).

There is therefore a desire to provide environmentally friendly antifouling ingredients. Enzymes are considered environmentally friendly since they will be degraded fast in the marine environment. Enzymes can therefore not be expected to be active over the life-time of a coating, but the self-polishing coating systems provide a solution to this problem. In a self-polishing coating, the enzymes will only be active and prone to degradation in the hydrated layer of the paint whereas the enzymes are expected to be quite stable in the inner dry layer of the paint, which provides a continuous source of fresh, active enzyme throughout the life-time of the paint.

The present invention alleviates the problem of the prior art In one aspect the present invention provides an anti-fouling composition comprising

(i) a surface coating material;
(ii) (a) a first enzyme; and
(b) a first substrate, wherein action of the first enzyme on the first substrate provides a second substrate
   (iil) a second enzyme, wherein the second enzyme is encapsulated in a silicate, and wherein said second enzyme generates an anti-foulant compound when acting on said second substrate.

In a further aspect the present invention provides a process for the preparation of a silicate encapsulated enzyme comprising the steps of

(i) providing a fermentation broth containing an enzyme or an enzyme isolated from a fermentation broth without drying
(ii) encapsulating the enzyme in a silicate.

In a further aspect the present invention provides a silicate encapsulated enzyme obtained or obtainable by the process of the present invention.

In a further aspect the present invention provides an anti-fouling composition comprising

(i) a surface coating material;
(ii) (a) a first enzyme; and
(b) a first substrate, wherein action of the first enzyme on the first substrate provides a second substrate
   (iil) a second enzyme, wherein the second enzyme is an enzyme prepared in accordance with the process of the present invention, and wherein said second enzyme generates an anti-foulant compound when acting on said second substrate.

In a further aspect the present invention provides a coating consisting of a composition according to the present invention.

In a further aspect the present invention provides a marine anti-foul consisting of a composition according to the present invention.

Aspects of the present invention are defined in the appended claims. These and other preferred aspects are discussed below.

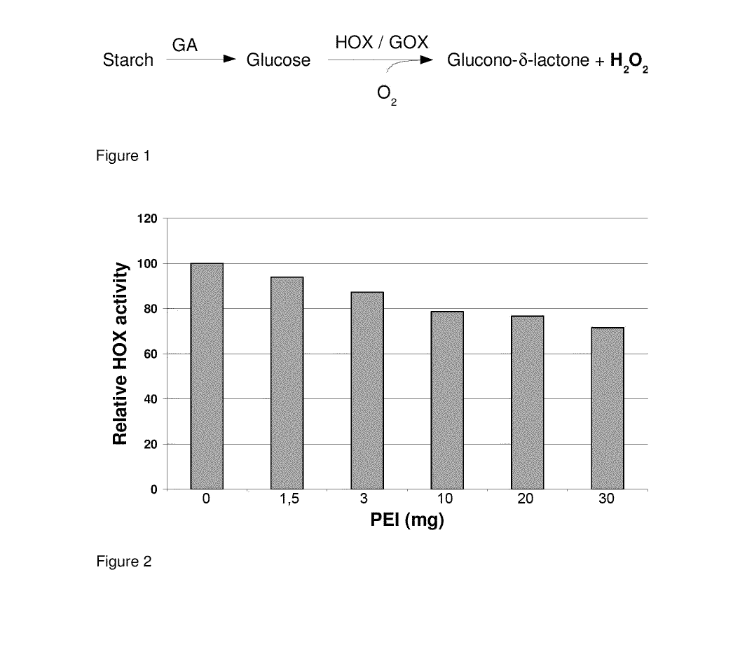

It has been found that systems of the prior art, such as that of WO-A-00/075293 (shown in FIG. 1) may be improved. In such systems the antifoulant hydrogen peroxide is produced by an oxidoreductase (for example Hexose oxidase (HOX) or Glucose oxidase (GOX)) from glucose and oxygen. Glucose is provided from the insoluble substrate (starch) by glucoamylase (GA). The present invention provides a system in which an enzyme coating stabilizes the enzyme components, for example Hexose oxidase (HOX) or Glucose oxidase (GOX). Consequently anti-foulant generating systems may be used in xylene based antifouling paint and at the same time retention of the enzyme in the paint film is improved.

The present invention provides a system in which the second enzyme is encapsulated by or in silica particles. Silica particles are hard and insoluble in both aqueous and organic solvents and thus provide an optimal encapsulation solution for enzymes in the anti fouling system described herein. The silicate encapsulated second enzyme is typically provided by providing a mixture of second enzyme and a polycationic polymer together with a phosphate buffered silicate solution (PBSi) resulting in the formation of a colloidal co-precipitate consisting of the enzyme and the polycation within a hydrated, amorphous silica matrix.

The antifouling composition is miscible with organic solvent in order to transfer the enzyme to an organic solvent based paint. This may be achieved by surfactants but the process is not very efficient. Alternatively the enzymes may be provided in a dry form. To date, the individual components have been mixed into the coating as e.g. glucoamylase (GA) in dry form, HOX/GOX in dry form and starch in dry form. While starch is commercially available as a powder, the industrially produced enzymes are primarily available as fermentation broth or concentrate, and each must first be dried individually to provide the powders used in previous art. Many carriers (e.g. salt or maltodextrin) used for making dry enzyme products are not compatible with paint. We have further provided an improved process in which mixing the first substrate (starch) and first enzyme (glucoamylase (GA)) in liquid form directly into the co-precipitation suspension, the formulation of dry particles is simplified to a one step drying procedure and reduced in cost and resources.

In-paint activity assays indicate a better performance for silica encapsulated enzymes, such as HOX, than non-encapsulated enzymes. Paint producing hydrogen peroxide based on starch/GA/Silica-(HOX/GOX) was successfully produced. In conclusion, silicate encapsulation, such as by co-precipitation, is very promising with respect to stabilisation and retention of enzymes in anti fouling paint.

In the present specification “foulants” referred to by the terms “anti-foul(s)”, “anti-fouling”, and “anti-foulants” include organisms which may reside and/or grow on the surface to be treated with the present composition. The organisms include micro-organisms such as bacteria, fungi and protozoa, and algae and organisms such as algae, plants and animals. The organism may be a marine organism.

In the present specification “anti-foulant” or “anti-fouling”, such as in anti-foulant compound” or “anti-fouling composition” refers to a material, or compound or composition which prevents or reduces or inhibits the growth of a foulant.

In the present specification “surface coating material” refers to a material, or compound or composition which adheres to a surface to provide a coating on the same. Surface coating materials are well known in the field of paints.

In the present specification a silicate is a material containing at least silicon and oxygen and comprising one or more Si—O—Si linkages. The scope of the term ‘silicate’ is well known to one skilled in the art and details can, for example, be found in Kirk-Othmer Encyclopedia of Chemical Technology (incorporated herein by reference).

The composition of the present invention comprises a first enzyme and a first substrate, wherein the first enzyme and the first substrate generate a substrate for the second enzyme of the present invention by action of the first enzyme on the first substrate. This combination of first enzyme and first substrate is herein after referred to as “substrate generator”.

### First Enzyme

Preferably, the first enzyme is selected from exo-acting enzymes capable of degrading oligomeric or polymeric substrates to monomeric units, e.g. β-galactosidase, peptidase; glucoamylase, and mixtures thereof.

Preferably the first enzyme is glucoamylase (EC 3.2.1.3). One skilled in the art will appreciate that glucoamylase is also known as amyloglucosidase.

Preferably the first enzyme is glucoamylase from Trichoderma reesei or glucoamylase from Humicola grisea.

Preferably the first enzyme is glucoamylase from Trichoderma reesei.

Preferably the first enzyme is glucoamylase from Trichoderma reesei prepared as described in US 2006/0094080.

Preferably the first enzyme is glucoamylase from Humicola grisea.

### First Substrate

The provision of a first substrate is advantageous because it provides for sustained and/or prolonged release of the second substrate by action of the first enzyme on the first substrate.

Preferably, the first substrate is selected from oligomers and polymers of substrates for oxidative enzymes, starch, lactose, cellulose, dextrose, peptide, inulin, and mixtures thereof.

In the two-step process preferably the first substrate is starch.

Native starch is particularly preferred as a first substrate. Native starch provides densely packed crystals which can be readily applied in a surface coatings. Moreover, Native starch is water insoluble.

Cellulose is also particularly preferred as a first substrate. Cellulose is a common component in paint and use of cellulose as a first substrate reduces the number of additional components which must be added to a paint composition.

### Second Enzyme

Preferably, the second enzyme is an oxidase. Preferably, the second enzyme is selected from glucose oxidase, L-amino acid oxidase, D-amino oxidase, galactose oxidase, hexose oxidase, pyranose oxidase, malate oxidase, cholesterol oxidase, arylalcohol oxidase, alcohol oxidase, lathosterol oxidase, aspartate oxidase, amine oxidase, D-glutamate oxidase, ethanolamine oxidase, NADH oxidase, urate oxidase (uricase) and mixtures thereof.

Preferably, the second enzyme is glucose oxidase, hexose oxidase or a mixture thereof.

Preferably, in one aspect the second enzyme is glucose oxidase.

Preferably, the first enzyme is glucose oxidase from Aspergillus niger.

Preferably, the first enzyme is glucose oxidase from Aspergillus niger and as prepared as described in U.S. Pat. No. 5,783,414.

Preferably, the first enzyme is glucose oxidase GC199 available from Genencor International Inc, Rochester, N.Y., USA.

Preferably, in one aspect the second enzyme is hexose oxidase.

Hexose oxidase (D-hexose: O₂-oxidoreductase, EC 1.1.3.5) (also referred to as HOX) is an enzyme that in the presence of oxygen is capable of oxidising D-glucose and several other reducing sugars including maltose, lactose and cellobiose to their corresponding lactones with subsequent hydrolysis to the respective aldobionic acids. Accordingly, HOX differs from another oxidoreductase, glucose oxidase, which can only convert D-glucose, in that the second enzyme can utilise a broader range of sugar substrates. The oxidation catalysed by HOX can be illustrated as follows:

D-glucose+O₂------>γ-D-gluconolactone+H₂O₂, or

D-galactose+O₂----->γ-D-galactonolactone+H₂O₂

As used herein, the term “HOX” denotes an enzyme which is capable of oxidising the substrates selected from the group consisting of D-glucose, D-galactose, D-mannose, maltose, lactose and cellobiose.

Preferably, the hexose oxidase is obtainable or is obtained from Chondrus cripus.

In one aspect the hexose oxidase enzyme is an enzyme covered by the disclosure of EP-A-0832245

Preferably, the first enzyme is Hexose oxidase from Chondrus crispus, used as fermentation broth and prepared as described in EP-A-0832245.

### Second Substrate

Preferably, the second substrate is selected from peptides, L-amino acid, and carbohydrates/sugars, including hexoses, preferably glucose, galactose, lactose, 2-deoxyglucose, pyranose, xylan, cellulose, inulin, starch, dextran, pectin, and mixtures thereof.

### Enzymes/Substrates

In a preferred embodiment the second enzyme/second substrate combination is selected from glucose/hexose oxidase, glucose/glucose oxidase, L amino acid/L amino acid oxidase, galactose/galactose oxidase, lactose/β-galactosidase/hexose oxidase, lactose/β-galactosidase/glucose oxidase, 2-deoxyglucose/glucose oxidase, pyranose/pyranose oxidase, and mixtures thereof.

In a highly preferred aspect the first substrate/first enzyme/second enzyme combination is starch/glucoamylase/hexose oxidase.

Preferably, the anti-fouling compound is hydrogen peroxide.

In one preferred aspect the first enzyme is present in an amount such that its activity is less than the activity of the second enzyme. Thus, the first enzyme will limit the rate of formation of anti-foulant compound. Thus in one preferred aspect the activity ratio of first enzyme:second enzyme is greater than 1:1, preferably at least 1:2, preferably at least 1:10, preferably at least 1:20, preferably at least 1:50, preferably at least 1:100, preferably at least 1:1000, preferably at least 1:10000.

In one preferred aspect the first enzyme is glucoamylase and the second enzyme is HOX or GOX and the activity ratio of glucoamylase to HOX/GOX is greater than 1:1, preferably at least 1:2, preferably at least 1:10, preferably at least 1:20, preferably at least 1:50, preferably at least 1:100, preferably at least 1:1000, preferably at least 1:10000.

In one preferred aspect the first enzyme is glucoamylase and the second enzyme is HOX and the activity ratio of glucoamylase to HOX is greater than 1:1, preferably at least 1:2, preferably at least 1:10, preferably at least 1:20, preferably at least 1:50, preferably at least 1:100, preferably at least 1:1000, preferably at least 1:10000.

In one preferred aspect the first enzyme is glucoamylase and the second enzyme is GOX and the activity ratio of glucoamylase to GOX is greater than 1:1, preferably at least 1:2, preferably at least 1:10, preferably at least 1:20, preferably at least 1:50, preferably at least 1:100, preferably at least 1:1000, preferably at least 1:10000.

### Encapsulation

Silica particles are hard and insoluble in both aqueous and organic solvents and are thus believed to provide an optimal encapsulation for enzymes in an anti fouling system. A procedure for co-precipitation of silicate and enzymes has previously been developed (and described in US2005/0158837). A mixture of enzyme and a polycationic polymer is mixed with a phosphate buffered silicate solution (PBSi) resulting in the formation of a colloidal co-precipitate consisting of the enzyme and the polycation within a hydrated, amorphous silica matrix.

Furthermore, the present invention provides a one-step formulation process which allows simplified formulation of the antifouling composition for paint.

In one preferred aspect the encapsulated second enzyme is a co-precipitate of an enzyme, a silicate and a N-containing organic template molecule. Preferably the N-containing organic template molecule is a polyamine, a modified polyamine, polyethyleneimine, a polypeptide or a modified polypeptide.

In one preferred aspect the encapsulated second enzyme is a co-precipitate of an enzyme, a silicate and a N-containing molecule selected from a polyamine, a modified polyamine, polyethyleneimine, a polypeptide and a modified polypeptide.

In one preferred aspect the silicate is obtained by neutralising an alkali metal silicate.

In one preferred aspect the encapsulated enzyme is obtained by or is obtainable by hydrolysing an organosilicate and adding a buffer.

In one preferred aspect the encapsulated second enzyme is a co-precipitate of an enzyme, a silicate and polyethyleneimine. Preferably the enzyme:polyethyleneimine ratio is from about 1 to about 20, such as 2 to about 15, or such as from about 5 to about 15, or such as from about 5 to about 10, or from 0.3 to about 10, or from about 0.5 to about 5, or from about 0.7 to about 2, or from about 0.75 to about 1.25.

In one preferred aspect the encapsulated second enzyme is a co-precipitate of an HOX, a silicate and polyethyleneimine. When the second enzyme is HOX the HOX:polyethyleneimine ratio may be from about 0.3 to about 10, more preferably from about 0.5 to about 5, more preferably from about 0.7 to about 2, more preferably from about 0.75 to about 1.25.

In one preferred aspect the encapsulated second enzyme is a co-precipitate of an GOX, a silicate and polyethyleneimine. When the second enzyme is GOX the GOX:polyethyleneimine ratio may from about 1 to about 20, more preferably from about 2 to about 15, more preferably from about 5 to about 15, more preferably from about 5 to about 10.

A silicate or organosilicate solution for encapsulating the enzyme may be prepared from silica precursors. For the purposes of the invention, a silicate precursor is an organic or inorganic substance that can give rise to silicon dioxide (Si02, silica) under selected conditions.

A silicate solution is a solution containing soluble silicon dioxide in the form of silicate or oligosilicate salts. The silicate solution used in the method is prepared by mixing a dilute alkali metal silicate solution or alkyl siliconate salt solution with an aqueous solution or an acidic resin to reduce the pH to 12 or lower to form a buffered silicate solution, such as a phosphate-buffered solution. The aqueous solution that reduces the pH of the alkali metal silicates or alkyl siliconate salts to 12 or lower, can be an acid, an acidic solution, or a low pH buffer. Acids useful for neutralization include phosphoric acid, citric acid, acetic acid, hydrochloric acid and the like. Weak acids such as phosphoric acid, citric acid, and acetic acid are preferred and phosphoric acid is more preferred. Acid resins useful for neutralization include Amberlite” ‘IR-120+, which is a strongly acidic cation exchanger, (Aldrich, Wis.).

Silicate precursors useful for the present invention include alkali metal silicates and alkyl siliconate salts. Alkali metal silicates include sodium silicates (e.g. sodium to metasilicate, sodium orthosilicate and sodium silicate solutions), potassium silicates, and cesium silicates. Preferred alkali metal silicates are sodium silicates and potassium silicates.

Preferred alkali metal silicates include sodium silicates. Sodium silicates are commercially available. For example, sodium metasilicate and sodium orthosilicate can be obtained from Gelest Inc. (Morrisville, Pa.). Sodium silicate solution (a solution of SiO and NaOH) can be obtained from Sigma Aldrich.

Alkyl siliconate salts include sodium alkyl siliconate, potassium alkyl siliconate, and cesium alkyl siliconate. Preferred alkyl siliconate salts are sodium alkyl siliconate and potassium alkyl siliconate. A preferred alkyl siliconate salt is sodium methyl siliconate. In this embodiment, the Si—OH groups capable of condensation with gel formation are generated by the protonation of Si—O-metal groups, such as an alkyl siliconate, e.g. sodium methylsiliconate, MeSi(ONa)₃.

The silicate solution may be prepared by first hydrolyzing tetraalkylortho-silicate with an acid, a base, or a catalyst, to form silicate sols. Silicate sols are defined as a stable colloidal solution of silicate oligomers where the particle size is in the nanometer range. Silicate sols can undergo gelation or precipitation when exposed to a change in pH or a catalyst (Iler, R. K. ‘The Chemistry of Silica’ (Wiley, 1979); Brinker, C. J. and Scherer, G. W.

‘Sol Gel Science: The Physics and Chemistry of Sol-Gel Processing’ (Academic press, 1990)). Silicate sols are then added to a buffer, an acid or a base to form a silicate solution having a pH of about 2 to about 12, more preferably about 4 to about 10 and most preferably about 5 to about 9. Examples of tetraalkylorthosilicates include tetramethylorthosilicate (TMOS) and tetraethylorthosilicate (TEOS).

An organosilicate solution is a solution containing soluble silicon dioxide in the form of silicate or oligosilicate salts and an organosilane, a silane containing at least one silicon-carbon (Si—C) bond. The organosilicate solution used in the method is prepared by first hydrolyzing a tetraalkylorthosilicate and one or more organosilanes selected from the group consisting of alkyltrialkoxysilane, aryltrialkoxysilane, dialkyldialkoxysilane, and diaryldialkoxysilane, to form sols at either an acidic pH (pH 1-6) or a basic pH (pH 8-13). A preferred acidic pH is, for example, pH 1-5, or pH 1.5-4. A preferred basic pH is, for example, pH 9-12. The sols are then added to a buffer, an acid, or a base to form an organosilicate solution having a pH of about 2 to about 12, preferably a pH of about 4 to about 10 and more preferably a pH of about 5 to about 9. For example, phenyltriethoxysilane (PTES) is hydrolyzed with an aqueous acid to form a phenylsilsesquioxane sol (PPSQ), which is combined with a silicate sol derived from a tetraethylorthosilicate and added to a buffer to form an organosilicate solution. The ratio of the organosilane to silicate precursor ranges from about 1:100 to about 10:1, preferably, about 1:50 to about 2:1 and preferably about 1:10 to about 1:1 on a molar basis.

### Process

As discussed herein, in one aspect the present invention provides a process for the preparation of a silicate encapsulated enzyme comprising the steps of

(i) providing a fermentation broth containing an enzyme or an enzyme isolated from a fermentation broth without drying
(ii) encapsulating the enzyme in a silicate.

In one aspect of the present process there is provided a fermentation broth containing an enzyme.

In one aspect of the present process there is provided an enzyme isolated from a fermentation broth without drying.

As will be appreciated from the description herein, the enzyme of the present process is preferably the second enzyme of the antifouling composition of the present invention.

In one aspect of the present process the process comprises the additional step (iii) of providing an additional enzyme (such as the first enzyme of the antifouling composition of the present invention)

In one aspect of the present process the process comprises the additional step (iii) of providing a substrate (such as the first substrate of the antifouling composition of the present invention).

In one aspect of the present process the process comprises the additional step (iii) of providing an additional enzyme (such as the first enzyme of the antifouling composition of the present invention) and a substrate (such as the first substrate of the antifouling composition of the present invention).

Preferably the additional enzyme (such as the first enzyme of the antifouling composition of the present invention) and a substrate (such as the first substrate of the antifouling composition of the present invention) are provided in ratios that results in a antifouling composition having the desired release of anti-foulant.

In one aspect of the present process the process comprises the additional step of (iv) drying the material. Such drying may provide a composition that may be mixed directly into an organic solvent based paint

In one aspect of the present process the process comprises the additional step (iii) defined herein and the additional step (iv) defined herein.

The present invention is advantageous in that crude liquid formulations of enzymes are combined in the same step (i.e. without intermediate drying or other separation steps) as the encapsulation of one component, thereby obtaining the appropriate ratios between components while significantly cutting costs of formulation from the point of view of enzyme manufacture as well as cutting needs for mixing facilities and production time from the point of view of coating formulation.

### Surface Coating Material

Preferably the surface coating material comprises components selected from polyvinyl chloride resins in a solvent based system, chlorinated rubbers in a solvent based system, acrylic resins and methacrylate resins in solvent based or aqueous systems, vinyl chloride-vinyl acetate copolymer systems as aqueous dispersions or solvent based systems, polyvinyl methyl ether, butadiene copolymers such as butadiene-styrene rubbers, butadiene-acrylonitrile rubbers, and butadiene-styrene-acrylonitrile rubbers, drying oils such as linseed oil, alkyd resins, asphalt, epoxy resins, urethane resins, polyester resins, phenolic resins, natural) rosin, rosin derivatives, disproportionated rosin, partly polymerised rosin, hydrogenated rosin, gum rosin, disproportionated gum rosin, non-aqueous dispersion binder systems, silylated acrylate binder systems, metal acrylate binder systems derivatives and mixtures thereof.

Preferably the surface coating material comprises a binder. Preferably the binder is selected from (natural) rosin, rosin derivatives, disproportionated rosin, partly polymerised rosin, hydrogenated rosin, gum rosin, disproportionated gum rosin, acrylic resins, polyvinyl methyl ether, vinyl acetate-vinychloride-ethylene terpolymers, non-aqueous dispersion binder systems, silylated acrylate binder systems and metal acrylate binder systems. Such binders are of particular interest for anti-fouling compositions used for marine purposes.

### Non-Aqueous Dispersion Binder System

The terms “non-aqueous dispersion resin” and similar expressions are intended to mean a shell-core structure that includes a resin obtained by stably dispersing a high-polarity, high-molecular weight resin particulate component (the “core component”) into a non-aqueous liquid medium in a low-polarity solvent using a high-molecular weight component (the “shell component”).

The non-aqueous dispersion resin may be prepared by a method wherein a polymerisable ethylenically unsaturated monomer which is soluble in a hydrocarbon solvent and which is polymerisable to form a polymer (the core component) which is insoluble in the hydrocarbon solvent, is subjected to dispersion polymerisation in accordance with a conventional method in the hydrocarbon solvent in the presence of a shell component (the dispersion stabiliser) made of a polymer which dissolves or swells in the solvent.

The non-aqueous dispersion-type resin utilised can be a resin known per se; or it can be produced like the known resins. Such non-aqueous dispersion-type resins and method for their preparation are described in, e.g., U.S. Pat. No. 3,607,821, U.S. Pat. No. 4,147,688, U.S. Pat. No. 4,493,914 and U.S. Pat. No. 4,960,828, Japanese Patent Publication No. 29,551/1973 and Japanese Laid-open Patent Application No. 177,068/1982. Specifically, as the shell component constituting the non-aqueous dispersion-type resin, various high-molecular substances soluble in a low-polarity solvent which are described in, e.g., U.S. Pat. No. 4,960,828 (Japanese Laid-open Patent Application No. 43374/1989), can be used.

From the aspect of antifouling property of the final paint coat, shell components such as an acrylic resin or a vinyl resin may be used.

As the core component, a copolymer of an ethylenically unsaturated monomer having a high polarity is generally applicable.

The non-aqueous dispersion-type resin can be formed by a method known per se. Examples thereof are a method in which the core component and the shell component are previously formed by block co-polymerization or graft co-polymerization, and they are then mixed in a low-polarity solvent and, if required, reacted to form a non-aqueous dispersion (see Japanese Patent Publication No. 29,551/1973), and a method in which a mixture of ethylenically unsaturated monomers at least one of which has a high-polarity group is co-polymerised in a solvent that dissolves the ethylenically unsaturated monomer but does not dissolve a polymer (core component) formed therefrom and in the presence of a dispersion stabiliser that either dissolves or stably disperses in said solvent, and if required, the obtained copolymer is further reacted with said dispersion stabiliser to afford a final non-aqueous dispersion (see U.S. Pat. No. 3,607,821 (Japanese Patent Publication No. 48,566/1982), Japanese Laid-open Patent Application No. 177,068/1982, No. 270,972/2001, No. 40,010/2001 and No. 37,971/2002). In the latter method, the dispersion stabiliser contains in a molecule the component soluble in the low-polarity solvent and the component having affinity for the resin being dispersed, or the dispersion stabiliser of the specific composition that dissolves in the low-polarity solvent is present as the shell component, and the component being dispersed as the core component is formed by copolymerisation of the monomers.

In the non-aqueous dispersion-type resin of the shell-core structure used in this invention, it is important that at least the core component has free acid groups or free acid groups and silyl ester groups that are convertible into the acid group by hydrolysis in sea water. Preferably 5-75% by weight, preferably 5-60% by weight, preferably 7-50% by weight, of the monomers of the core polymer should carry free acid groups. As the free acid groups will have direct influence on the properties of the paint formulation, whereas the silyl ester groups will only have influence after hydrolysis in seawater, it is important that no more than 3% by weight of the monomers of the core component are silyl ester monomers. Typically, no more than 1% by weight of monomers of the core component are silyl ester monomers, and most often no silyl ester groups are present in the core.

Examples of silyl ester monomers are silyl esters of acrylic or methacrylic acid.

If desired, a smaller proportion of the free acid groups or silyl ester groups may also be contained in the shell component. It is, however, believed that less than 3% by weight of the monomers of shell component are free acid groups or silyl ester groups.

The expression “free acid group” is intended to cover the acid group in the acid form. It should be understood that such acid groups temporarily may exist on salt form if a suitable counter ion is present in the composition or in the environment. As an illustrative example, it is envisaged that some free acid groups may be present in the sodium salt form if such groups are exposed to salt water.

Thus, the non-aqueous dispersion-type resin preferably has a resin acid value of 15-400 mg KOH/g, preferably 15 to 300 mg KOH/g, preferably 18 to 300 mg KOH/g. If the total acid value of the non-aqueous dispersion resin is below 15 mg KOH/g, the polishing rate of the paint coat may be too low and the antifouling property will often be unsatisfactory. On the other hand, if the total acid value is above 400 mg KOH/g, the polishing rate may be too high for that reason a problem of water resistance (durability of the paint coat in seawater) becomes a problem. (When the core component and/or the shell component contain the acid precursor group, the resin acid value is one given after the group is converted into the acid group by hydrolysis). The “resin acid value” here referred to is an amount (mg) of KOH consumed to neutralise 1 g of a resin (solids content), expressing a content of an acid group (in case of the acid precursor group, a content of an acid group formed by hydrolysis) of the resin (solids content).

It is advisable that the acid group and/or the acid precursor group is contained in the core component such that the content thereof is, as a resin acid value, at least 80%, preferably at least 90%, more preferably at least 95% of the total resin acid value of the non-aqueous dispersion-type resin.

If the acid value in the core component of the non-aqueous dispersion resin is below 80% of the total acid value of the non-aqueous dispersion-type resin, i.e. the acid value of the shell component is above 20% of the total acid value, potential problems may be as described above with respect to water resistance and durability. Furthermore, if the coating composition comprises free metal ions, a problem with respect to gelation may occur if the acid value of the shell component is above 20% of the total acid value.

This being said, it is normally preferred that the shell component is hydrophobic.

The dry weight ratio of the core component to the shell component in the non-aqueous dispersion-type resin is not especially limited, but is normally in the range of 90/10 to 10/90, preferably 80/20 to 25/75, preferably 60/40 to 25/75.

Furthermore, it is believed that the dry matter of the non-aqueous dispersion resin normally constitutes in the range of 2-30%, preferably 4-25%, preferably 5-25%, preferably 5-20% by wet weight of the coating composition.

As the solvent for dispersing the non-aqueous dispersion resin that will be a binder, various organic solvents that are commonly used for paints can be used without any particular restrictions.

Examples of solvents in which the components of the non-aqueous dispersion resin paint composition are dissolved or dispersed are alcohols such as methanol, ethanol, propanol, isopropanol, butanol, isobutanol and benzyl alcohol; alcohol/water mixtures such as ethanol/water mixtures; aliphatic, cycloaliphatic and aromatic hydrocarbons such as white spirit, cyclohexane, toluene, xylene and naphtha solvent; ketones such as methyl ethyl ketone, acetone, methyl isobutyl ketone, methyl isoamyl ketone, diacetone alcohol and cyclohexanone; ether alcohols such as 2-butoxyethanol, propylene glycol monomethyl ether, ethylene glycol monoethyl ether, ethyl ether and butyl diglycol; esters such as ethyl acetate, propyl acetate, methoxypropyl acetate, n-butyl acetate and 2-ethoxyethyl acetate; chlorinated hydrocarbons such as methylene chloride, tetrachloroethane and trichloroethylene; and mixtures thereof.

Useful solvents are in particular hydrocarbon type solvents and include aliphatic, alicyclic and aromatic solvents. In the present invention, it is preferred to employ an aliphatic hydrocarbon solvent and/or an alicyclic hydrocarbon solvent, or such a solvent in the major amount.

Suitable aliphatic and alicyclic hydrocarbon solvents include, for example, n-hexane, iso-hexane, n-heptane, n-octane, iso-octane, n-decane, n-dodecane, cyclohexane, methylcyclohexane and cycloheptane. Commercial products include, for example, mineral spirit ec, vm&p naphtha and shellzole 72 (manufactured by Shell Chemical Co.); naphtha no. 3, naphtha no. 5, naphtha no. 6 and solvent no. 7 (manufactured by Exxon Chemical Co.); ip solvent 1016, ip solvent 1620 and ip solvent 2835 (manufactured by Idemitsu Petrochemical co., ltd.); and pengazole an-45 and pengazole 3040 (manufactured by Mobile Oil Co.).

Further, the aromatic solvents include, for example, benzene, toluene, xylene and decalin. Commercial products include, for example, Solvesso 100 and Solvesso 150 (manufactured by Exxon Chemical Co.); and Swazole (manufactured by Maruzen Oil Co., Ltd.).

These hydrocarbon type solvents may be used alone or in combination as a mixture of two or more of them.

### Silylated Acrylate Binder System

In a further aspect, the co-polymer to be used in the coating composition comprises at least one side chain bearing at least one terminal group of the general formula I:

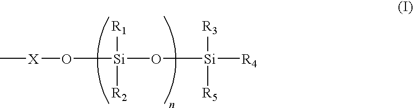

wherein n is an integer of 1 or more.

When n is an integer of 1, 2, 3, 4 or more, it is in these cases preferred that n is up to about 5,000, preferably n is an integer from 1-50, preferably n is an integer from 2-15.

X is selected from:

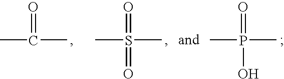

R₁-R₅ are each groups independently selected from the group consisting of C₁₋₂₀-alkyl, C₁₋₂₀-alkoxy, phenyl, optionally substituted phenyl, phenoxy and optionally substituted phenoxy. With respect to the above formula I it is generally preferred that each of the alkyl and alkoxy groups has up to about 5 carbon atoms (C₁₋₅-alkyl). Illustrative examples of substituents for the substituted phenyl and phenoxy groups include halogen, C₁₋₅-alkyl, C₁₋₅-alkoxy or C₁₋₁₀-alkylcarbonyl. As indicated above, R₁-R₅ may be the same or different groups.

Monomers comprising the terminal groups of the general formula I above may be synthesised as described in EP 0 297 505 B1, i.e. the monomers may, for example, be synthesised by condensation, such as e.g. dehydrocondensation of e.g. acrylic acid, methacrylic acid or a maleic acid monoester with an organosilyl compound having R₃-R₅ in its molecule, such as an organosiloxane having a di-substituted monohydroxysilane group at one terminal, a tri-substituted monohydroxysilane, an organosiloxane having a hydroxymethyl group or a halogen methyl group, such as a chloro methyl group, at one terminal, or a tri-substituted silane.

Such monomers may be co-polymerised (in order to obtain the co-polymer to be used in the coating composition according to the invention) with a vinyl polymerisable monomer A. Examples of suitable vinyl polymerisable monomers include methacrylate esters such as methyl methacrylate, ethyl methacrylate, butyl methacrylate, 2-ethylhexyl methacrylate, 2-hydroxyethyl methacrylate and methoxy ethyl methacrylate; acrylate esters such as ethyl acrylate, butyl acrylate, 2 ethylhexyl acrylate and 2-hydroxyethyl acrylate; maleic acid esters such as dimethyl maleate and diethyl maleate; fumaric acid esters such as dimethyl fumarate and diethyl fumarate; styrene, vinyltoluene, α-methylstyrene, vinyl chloride, vinyl acetate, butadiene, acrylamide, acrylonitrile, methacrylic acid, acrylic acid, isobornyl methacrylate and maleic acid.

These vinyl polymerisable monomers (A) act as modifying components that impart desirable properties to resulting co-polymer. These polymers are also useful for the purpose of obtaining polymers that have higher molecular weights than the homopolymers made up of monomers comprising the terminal group of the general formula II and III (below). The amount of vinyl polymerisable monomers is not more than 95% by weight of the total weight of the resulting co-polymer, preferably not more than 90% by weight. Accordingly, the amount of monomers comprising the terminal groups of the general formula I above is at least 5% by weight, in particular at least 10% by weight.

The co-polymers comprising at least one side chain bearing at least one terminal group of the general formula I (shown above) may be formed by polymerising at least one monomer comprising a terminal group of the general formula I with one or more of the vinyl polymerisable monomers (A) above in the presence of a suitable (vinyl) polymerisation initiator in accordance with routine procedures. Methods of polymerisation include solution polymerisation, bulk polymerisation, emulsion polymerisation, suspension polymerisation, anionic polymerisation and co-ordination polymerisation. Examples of suitable vinyl polymerisation initiators are azo compounds such as azobisisobutyronitrile and triphenylmethylazobenzene, and peroxides such as benzoyl peroxide and di-tert-butyl peroxide.

The co-polymers to be prepared by the methods described above preferably have weight average molecular weights in the range of 1,000-1,500,000, such as in the range of 5,000-1,500,000, e.g. in the range of 5,000-1,000,000, in the range of 5,000-500,000, in the range of 5,000-250,000, or in the range of 5,000-100,000. If the molecular weight of the co-polymer is too low, it is difficult to form a rigid, uniform and durable film. If, on the other hand, the molecular weight of the co-polymer is too high, it makes the varnish highly viscous. Such a high viscosity varnish should be thinned with a solvent for formulation of a coating composition. Therefore, the resin solids content of the coating composition is reduced and only a thin dry film can be formed by a single application. This is inconvenient in that several applications of the coating composition are necessary to attain proper dry film thickness.

Although a number of different methods for determining the weight average molecular weight of the polymer in question will be known to the person skilled in the art, it is preferred that the weight average molecular weight is determined in accordance with the GPC-method described at page 34 in WO 97/44401.

In another aspect, the co-polymer to be used in the coating composition comprises at least one side chain bearing at least one terminal group of the general formula II:

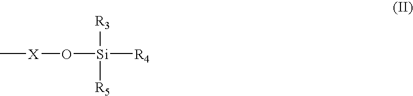

wherein X, R₃, R₄ and R₅ are as defined for general formula I.

Examples of monomers having a terminal group of the general formula II (shown above) are acid functional vinyl polymerisable monomers, such as monomers derived from acrylic acid, methacylic acid, maleic acid (preferably in the form of a monoalkyl ester with 1-6 carbon atoms) or fumaric acid (preferably in the form of a monalkyl ester with 1-6 carbon atoms).

With respect to the triorganosilyl group, i.e. the —Si(R₃)(R₄)(R₅) group, shown in the above formulae I or II, R₃, R₄ and R₅ may be the same or different, such as C₁₋₂₀-alkyl (e.g. methyl, ethyl, propyl, butyl, cycloalkyl such as cyclohexyl and substituted cyclohexyl); aryl (e.g., phenyl and naphthyl) or substituted aryl (e.g., substituted phenyl and substituted naphthyl). Examples of substituents for aryl halogen, C₁₋₁₈-alkyl, acyl, sulphonyl, nitro, or amino.

Thus, specific examples of a suitable triorganosilyl group (i.e. the —Si(R₃)(R₄)(R₅) group) shown in the general formula I or II include trimethylsilyl, triethylsilyl, tri-n-propylsilyl, tri-n-butylsilyl, tri-iso-propylsilyl, tri-n-pentylsilyl, tri-n-hexylsilyl, tri-n-octylsilyl, tri-n-dodecylsilyl, triphenylsilyl, tri-p-methylphenylsilyl, tribenzylsilyl, tri-2-methylisopropylsilyl, tri-tert-butylsilyl, ethyldimethylsilyl, n-butyldimethylsilyl, di-iso-propyl-n-butylsilyl, n-octyl-di-n-butylsilyl, di-iso-propryloctadecylsilyl, dicyclohexylphenylsilyl, tert-butyldiphenylsilyl, dodecyldiphenylsilyl and diphenylmethylsilyl.

Specific examples of suitable methacrylic acid-derived monomers bearing at least one terminal group of the general formula I or II include trimethylsilyl (meth)acrylate, triethylsilyl(meth)acrylate, tri-n-propylsilyl(meth)acrylate, triisopropylsilyl (meth)acrylate, tri-n-butylsilyl (meth)acrylate, triisobutylsilyl (meth)acrylate, tri-tert-butylsilyl(meth)acrylate, tri-n-amylsilyl (meth)acrylate, tri-n-hexylsilyl (meth)acrylate, tri-n-octylsilyl (meth)acrylate, tri-n-dodecylsilyl (meth)acrylate, triphenylsilyl (meth)acrylate, tri-p-methylphenylsilyl (meth)acrylate, tribenzylsilyl (meth)acrylate, ethyldimethylsilyl (meth)acrylate, n-butyldimethylsilyl (meth)acrylate, diisopropyl-n-butylsilyl (meth)acrylate, n-octyldi-n-butylsilyl (meth)acrylate, diisopropylstearylsilyl (meth)acrylate, dicyclohexylphenylsilyl (meth)acrylate, t-butyldiphenylsilyl (meth)acrylate, and lauryldiphenylsilyl (meth)acrylate.

Specific examples of suitable maleic acid-derived and fumaric acid-derived monomers bearing at least one terminal group of the general formula I or II include triisopropylsilyl methyl maleate, triisopropylsilyl amyl maleate, tri-n-butylsilyl n-butyl maleate, tert-butyldiphenylsilyl methyl maleate, t-butyldiphenylsilyl n-butyl maleate, triisopropylsilyl methyl fumarate, triisopropylsilyl amyl fumarate, tri-n-butylsilyl n-butyl fumarate, tert-butyldiphenylsilyl methyl fumarate, and tert-butyldiphenylsilyl n-butyl fumarate.

In another aspect, the co-polymer to be used in the coating composition comprises monomer units with a terminal group of the general formula II (as discussed above) in combination with a second monomer B of the general formula III:

Y—(CH(R_{A})—CH(R_{B})—O)ₚ-Z (III)

wherein Z is a C₁₋₂₀-alkyl group or an aryl group; Y is an acryloyloxy group, a methacryloyloxy group, a maleinoyloxy group or a fumaroyloxy group; R_{A} and R_{B} are independently selected from the group consisting of hydrogen, C₁₋₂₀-alkyl and aryl; and p is an integer of 1 to 25.

If p>2, R_{A} and R_{B} are preferably hydrogen or CH₃, i.e. if p>2 the monomer B is preferably derived from a polyethylene glycol or a polypropylene glycol.

If p=1 it is contemplated that monomers, wherein R_{A} and R_{B} are larger groups, such as C₁₋₂₀-alkyl or aryl.

As shown in formula III, monomer B has in its molecule an acryloyloxy group, a methacryloyloxy group, a maleinoyloxy group (preferably in the form of a mono-C₁₋₆-alkyl ester), or a fumaroyloxy group (preferably in the form of a mono-C₁₋₆-alkyl ester) as an unsaturated group (Y) and also alkoxy- or aryloxypolyethylene glycol. In the alkoxy- or aryloxypolyethylene glycol group, the degree of polymerisation (p) of the polyethylene glycol is from 1 to 25.

Examples of the alkyl or aryl group (Z) include C₁₋₁₂-alkyl (e.g., methyl, ethyl, propyl, butyl, cycloalkyl such as cyclohexyl and substituted cyclohexyl); and aryl (e.g., phenyl and naphthyl) and substituted aryl (e.g., substituted phenyl and substituted naphthyl).

Examples of substituents for aryl include halogen, C₁₋₁₈-alkyl group, C₁₋₁₀-alkylcarbonyl, nitro, or amino.

Specific examples of monomer B which has a (meth)acryloyloxy group in a molecule include methoxyethyl (meth)acrylate, ethoxyethyl (meth)acrylate, propoxyethyl (meth)-acrylate, butoxyethyl (meth)acrylate, hexoxyethyl (meth)acrylate, methoxydiethylene glycol (meth)acrylate, methoxytriethylene glycol (meth)acrylate, ethoxydiethylene glycol (meth)acrylate, and ethoxytriethylene glycol (meth)acrylate.

Specific examples of monomer B which has a maleinoyloxy or fumaroyloxy group in a molecule include methoxyethyl n-butyl maleate, ethoxydiethylene glycol methyl maleate, ethoxytriethylene glycol methyl maleate, propoxydiethylene glycol methyl maleate, butoxyethyl methyl maleate, hexoxyethyl methyl maleate, methoxyethyl n-butyl fumarate, ethoxydiethylene glycol methyl fumarate, ethoxytriethylene glycol methyl fumarate, propoxydiethylene glycol methyl fumarate, butoxyethyl methyl fumarate, and hexoxyethyl methyl fumarate.

As will be understood by the person skilled in the art, other vinyl monomers may be incorporated in the resulting co-polymer comprising either monomer units having a terminal group of the general formula II (shown above) or in the resulting co-polymer comprising monomer units having a terminal group of the general formula II (shown above) in combination with the second monomer B of the formula III (shown above).

With respect to other monomers co-polymerisable with the above-mentioned monomers, use may be made of various vinyl monomers such as the vinyl polymerisable monomers (A) discussed above.

In the monomer mixture, the proportions of monomer having a terminal group of the general formula II, monomer B and other monomer(s) co-polymerisable therewith (e.g. monomer A) may be suitably determined depending on the use of the coating composition. In general, however, it is preferred that the proportion of the monomer having a terminal group of the general formula II is from 1-95% by weight, that of monomer B is from 1-95% by weight, and that of other monomer(s) co-polymerisable therewith is from 0-95% by weight on the basis of the total weight of the monomers.

Thus, co-polymers comprising a combination of monomer units bearing a terminal group of the general formula II and monomer units B (and optionally monomer A) can be obtained by polymerising such monomer mixtures in the presence of a vinyl polymerisation initiator by any of various methods such as solution polymerisation, bulk polymerisation, emulsion polymerisation, and suspension polymerisation in an ordinary way, which will be known to the person skilled in polymer chemistry. It is preferred, however, to employ the solution polymerisation method or the bulk polymerisation method.

Examples of the vinyl polymerisation initiators include azo compounds such as azobis-isobutyronitrile and triphenylmethylazobenzene; and peroxides such as benzoyl to peroxide, di-tert-butyl peroxide, tert-butyl peroxybenzoate, and tert-butyl peroxyisopropylcarbonate.

The molecular weight of the resulting co-polymer thus obtained is desirably in the range of 1,000-150,000, preferably in the range of 3,000-100,000, preferably in the range of 5,000-100,000 in terms of weight-average molecular weight. Too low molecular weights result in difficulties in forming normal coating film, while too high molecular weights result in disadvantages that a single coating operation only gives thin coating film and, hence, coating operations should be conducted in a larger number. It is preferred to regulate the solid content of the polymer solution to a value in the range of 5-90% by weight, desirably from 15-85% by weight.

In a further aspect, the co-polymer to be used in the coating composition comprises monomer units with a terminal group of the general formula II (as discussed above) in combination with a second monomer C of the general formula IV:

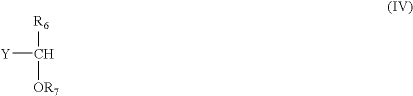

wherein Y is an acryloyloxy group, a methacryloyloxy group, a maleinoyloxy group or a fumaroyloxy group, and both of R₆ and R, are C₁₋₁₂-alkyl.

As shown in formula IV, monomer C has in its molecule an acryloyloxy group, a meth-acryloyloxy group, a maleinoyloxy group (preferably in the form of a mono-C₁₋₆-alkyl ester), or a fumaroyloxy group (preferably in the form of a mono-C₁₋₆-alkyl ester) as an unsaturated group (Y) and also a hemi-acetal group.

In the hemi-acetal group, examples of R₆ include C₁₋₁₂-alkyl, preferably C₁₋₄-alkyl (e.g., methyl, ethyl, n-propyl, n-butyl, isopropyl, isobutyl, and tert-butyl); and examples of R₇ include C₁₋₁₂-alkyl, preferably C₁₋₈-alkyl (e.g., methyl, ethyl, n-propyl, isopropyl, n-butyl, isobutyl, and tert-butyl), and a substituted or unsubstituted C₅₋₈-cycloalkyl (e.g., cyclohexyl).

Monomer C can be prepared by an ordinary addition reaction of a carboxy group-containing vinyl monomer selected from acrylic acid, methacrylic acid, maleic acid (or monoester thereof), and fumaric acid (or monoester thereof), with an alkyl vinyl ether (e.g., ethyl vinyl ether, propyl vinyl ether, butyl vinyl ether, hexyl vinyl ether, and 2-ethylhexyl vinyl ether), or a cycloalkyl vinyl ether (e.g., cyclohexyl vinyl ether).

As will be understood by the person skilled in the art, other vinyl monomers may be incorporated in the resulting co-polymer comprising monomer units having a terminal group of the general formula II (shown above) in combination with the second monomer C of the formula IV (shown above).

With respect to other monomers co-polymerisable with the above-mentioned monomers, use may be made of various vinyl monomers such as the vinyl polymerisable monomers (A) discussed above.

In the monomer mixture, the proportions of monomer having a terminal group of the general formula II, monomer C and other monomer(s) co-polymerisable therewith (e.g. monomer A) may be suitably determined depending on the use of the coating composition. In general, however, it is preferred that the proportion of the monomer having a terminal group of the general formula II is from 1-95% by weight (preferably from 1-80% by weight), that of monomer C is from 1-95% by weight (preferably from 1-80% by weight), and that of other monomer(s) co-polymerisable therewith is up to 98% by weight on the basis of the total weight of the monomers.

Thus, co-polymers comprising a combination of monomer units bearing a terminal group of the general formula II and monomer units C (and optionally monomer A) can be obtained by polymerising such monomer mixtures in the presence of a vinyl polymerisation initiator by any of various methods such as solution polymerisation, bulk polymerisation, emulsion polymerisation, and suspension polymerisation in an ordinary way, which will be known to the person skilled in polymer chemistry. It is preferred, however, to employ the solution polymerisation method or the bulk polymerisation method.

Examples of the vinyl polymerisation initiators include azo compounds such as azobisisobutyronitrile and triphenylmethylazobenzene; and peroxides such as benzoyl peroxide, di-tert-butyl peroxide, tert-butyl peroxybenzoate, and tert-butyl peroxyisopropylcarbonate.

The molecular weight of the resulting co-polymer thus obtained is desirably in the range of 1,000-150,000, preferably in the range of 3,000-100,000, preferably in the range of 5,000-100,000 in terms of weight-average molecular weight. Too low molecular weights result in difficulties in forming normal coating film, while too high molecular weights result in disadvantages that a single coating operation only gives thin coating film and, hence, coating operations should be conducted in a larger number.

Although it is preferred that the chemistry of the binder co-polymer is as described above, it is contemplated that also other silyl-containing co-polymers having a slight different structure may be useful for the purposes described herein. Thus, an example of a binder co-polymer having a slight different structure compared to the chemistry disclosed above is a binder co-polymer comprising at least one side chain bearing at least one terminal group of formula V:

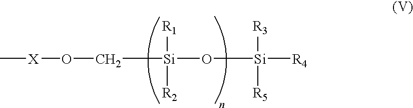

wherein X, n, R₁, R₂, R₃, R₄ and R₅ are as defined above in connection with general formula I.

### Metal Acrylate Binder System

In another aspect, the co-polymer to be used in the coating composition according to the invention comprises at least one side chain bearing at least one terminal group of the general formula VI

—X—O-M-(L)ₙ (VI)

wherein X is selected from:

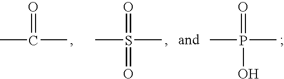

Wherein n is as defined above with regard to general formula I.

M is a metal. Metal (M) is any metal having a valency of 2 or more may be used. Specific examples of suitable metals may be selected from Ca, Mg, Zn, Cu, Ba, Te, Pb, Fe, Co, Ni, Bi, Si, Ti, Mn, Al and Sn. Preferred examples are Co, Ni, Cu, Zn, Mn, and Te, in particular Cu and Zn. When synthesising the metal-containing co-polymer, the metal may be employed in the form of its oxide, hydroxide or chloride. It is contemplated, however, that the metal may also be employed in the form of other halogenides (such as its fluoride, iodide or bromide salt) or in the form of its sulfide or carbonate.

L is a ligand.

Examples of monomers having a terminal group of the general formulae I or II (shown above) are acid-functional vinyl polymerisable monomers, such as methacrylic acid, acrylic acid, p-styrene sulfonic acid, 2-methyl-2-acrylamide propane sulfonic acid, methacryl acid phosphoxy propyl, methacryl 3-chloro-2-acid phosphoxy propyl, methacryl acid phosphoxy ethyl, itaconic acid, maleic acid, maleic anhydride, monoalkyl itaconate (e.g. methyl, ethyl, butyl, 2-ethyl hexyl), monalkyl maleate (e.g. methyl, ethyl, butyl, 2-ethyl hexyl; half-ester of acid anhydride with hydroxyl containing polymerisable unsaturated monomer (e.g. half-ester of succinic anhydride, maleic anhydride or phthalic anhydride with 2-hydroxy ethyl methacrylate.

As will be understood by the person skilled in the art, and as discussed in detail below, the above-mentioned monomers may be co-polymerised (in order to obtain the co-polymer to be used in the coating composition according to the invention) with one or more vinyl polymerisable monomers. Examples of such vinyl polymerisable monomers are methyl acrylate, methyl methacrylate, ethyl acrylate, ethyl methacrylate, propyl acrylate, propyl methacrylate, butyl acrylate, butyl methacrylate, octyl acrylate, octyl methacrylate, 2-ethyl hexyl acrylate, 2-ethyl hexyl methacrylate, methoxy ethyl methacrylate, styrene, vinyl toluene, vinyl pyridine, vinyl pyrolidone, vinyl acetate, acrylonitrile, methacrylonitrile, dimethyl itaconate, dibutyl itaconate, di-2-ethyl hexyl itaconate, dimethyl maleate, di (2-ethyl hexyl) maleate, ethylene, propylene and vinyl chloride.

With respect to the ligand (L), each individual ligand is preferably selected from the group consisting of

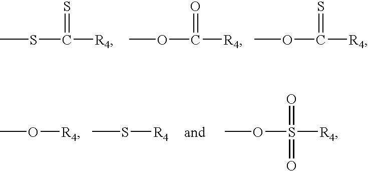

wherein R₄ is a monovalent organic residue.

Preferably, R₄ is selected from the group consisting of

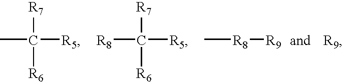

wherein R₅ is hydrogen or a hydrocarbon group having from 1 to 20 carbon atoms; R₆ and R, each independently represents a hydrocarbon group having from 1 to 12 carbon atoms;
R₈ is a hydrocarbon group having from 1 to 4 carbon atoms; and R₉ is cyclic hydrocarbon group having from 5 to 20 carbon atoms, such as abietic acid, pallustric acid, neoabietic acid, levopimaric acid, dehydroabietic acid, pimaric acid, isopimaric acid, sandaracopimaric acid and Δ8,9-isopimaric acid.

Examples of compounds which may be used as ligands (L) are:

(1) Compounds comprising the group

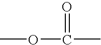

e.g. aliphatic acids, such as levulinic acid; alicyclic acids, such as naphthenic acid, chaulmoogric acid, hydnocarpusic acid, neo abietic acid, levo pimaric acid, palustric acid, 2-methyl-bicyclo-2,2,1-heptane-2-carboxylic acid; aromatic carboxylic acids such as salicylic acid, cresotic acid, α-naphthoic acid, β-naphthoic acid, p-oxy benzoic acid; halogen containing aliphatic acids, such as monochloro acetic acid, monofluoro acetic acid; halogen containing aromatic acids, such as 2,4,5-trichloro phenoxy acetic acid, 2,4-dichloro phenoxy acetic acid, 3,5-dichloro benzoic acid; nitrogen-containing organic acids, such as quinoline carboxylic acid, nitro benzoic acid, dinitro benzoic acid, nitronaphthalene carboxylic acid; lactone carboxylic acids, such as pulvinic acid, vulpinic acid; uracil derivatives, such as uracil-4-carboxylic acid, 5-fluoro uracil-4-carboxylic acid, uracil-5-carboxylic acid; penicillin-derived carboxylic acids, such as penicillin V, ampicillin, penicillin BT, penicillanic acid, penicillin G, penicillin 0; Rifamycin B, Lucensomycin, Salcomycin, chloroamphenicol, variotin, Trypacidine; and various synthetic fatty acids.
(2) Compounds comprising the group

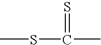

e.g. dimethyl dithiocarbamate and other dithiocarbamates.

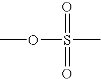

(3) Compounds comprising the group
e.g. sulphur containing aromatic compounds, such as 1-naphthol-4-sulphonic acid, p-phenyl benzene sulphonic acid, β-naphthalene sulphonic acid and quinoline sulphonic acid.
(4) Compounds comprising the group

—S—

such as compounds comprising the following groups
(5) Compounds comprising the group

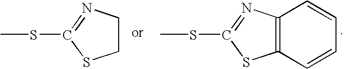

such as various thiocarboxylic compounds.

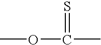

(6) Compounds comprising the group —O— or —OH
e.g. phenol, cresol, xylenol, thymol, carvacol, eugenol, isoeugenol, phenyl phenol, benzyl phenol, guajacol, butyl stilbene, (di) nitro phenol, nitro cresol, methyl salicylate, benzyl salicylate, mono-, di-, tri-, tetra- and penta-chlorophenol, chlorocresol, chloroxylenol, chlorothymol, p-chloro-o-cyclo-hexyl phenol, p-chloro-o-cyclopentyl phenol, p-chloro-o-n-hexyl phenol, p-chloro-o-benzyl phenol, p-chloro-o-benzyl-m-cresol and other phenols; β-naphthol, 8-hydroxy quinoline.

Although not generally preferred, it is also possible that one or more or all of the ligands (L) are —OH groups.

The co-polymer to be used in the coating composition according to the invention may be prepared as described in e.g. EP 0 471 204 B1, EP 0 342 276 B1 or EP 0 204 456 B1, i.e. by one of the following methods:

A method wherein a polymerisable unsaturated monomer having the desired organic acid metal ester bond at an end portion is first prepared and co-polymerised with other polymerisable unsaturated monomer(s);

A method wherein a co-polymer obtained by the co-polymerisation of a polymerisable unsaturated organic acid monomer with other polymerisable unsaturated monomer(s) is reacted with a monovalent organic acid and a metal oxide, chloride or hydroxide or is subjected to an ester exchange reaction with a monovalent carboxylic acid metal ester.

More specifically, the co-polymer may be prepared by either one of the following methods.

(1) A mixture of
(a) a metal oxide, hydroxide, sulfide or chloride,
(b) a monovalent organic acid or its alkali metal salt, and
(c) a polymerisable unsaturated organic acid or its alkali metal salt, is heated under stirring at a temperature lower than the decomposition temperature of the desired metal ester product, and the by-produced substances as alkali metal chloride, water, monovalent organic acid metal ester; bifunctional polymerizable unsaturated organic acid metal salt are removed to obtain a purified metal ester between the polymerisable unsaturated organic acid and the monovalent organic acid.

In the above-mentioned reaction, it is not always necessary to use stoichiometric amounts of (a), (b) and (c) and one may use, in terms of equivalent ratio, (a):(b):(c)=1:0.8-3:0.8-2 to obtain the desired product.

The thus obtained metal ester between the polymerisable unsaturated organic acid and the monovalent organic acid or the mixture of said metal ester and the monovalent organic metal ester is then subjected to a homo-polymerisation or a co-polymerisation with other co-polymerisable monomer(s) to give the desired co-polymer having at least one side chain bearing at least terminal group as shown in formulae I or II above.

(2) Alternatively, a mixture of

(d) a co-polymer having at a side chain an organic acid or its alkali metal salt,
(e) a metal oxide, hydroxide, sulfide or chloride, and
(f) a monovalent organic acid, is heated under stirring at a temperature lower than the decomposition temperature of the desired metal ester-containing co-polymer, and the by-produced substances are removed, if desired, to obtain a co-polymer having at least one side chain bearing at least one terminal group as shown in formulae I or II above.

With respect to the ratios of the materials used in this reaction, it is preferred to use, in terms of equivalent ratio, (d):(e):(f)=1:0.8-1.5:0.8-2 and more preferably 1:1.0-1.2:1.0-1.5.

When a low boiling monovalent organic acid is selected and the reaction is accompanied by a dehydration, there is a fear that the monovalent organic acid is distilled off together with water and that a metal bond is formed between the polymer-chains, thereby causing an increase in viscosity and gelation of the product. Therefore, in this particular case, it is therefore preferred to use a higher amount of (f) than indicated above.

(3) Alternatively, the desired product may be prepared by reacting a co-polymer having at a side chain an organic acid (g) with a monovalent organic acid metal ester (h) at a temperature of not higher than the decomposition temperature of the desired product, thereby effecting an ester exchange reaction between the materials used.

In this reaction, when the selected monovalent organic acid has a low boiling point (as, for example, acetic acid), there is a fear that a metal ester bonding is formed between the polymer-chains and, therefore, the reaction should be carefully controlled and proceeded with. Usually, the material (h) is used in an amount of from 0.3 to 3 equivalents, more preferably of from 0.4 to 2.5 equivalents, per equivalent of (g).

Examples of polymerisable unsaturated organic acids (c) to be used include methacrylic acid, acrylic acid, p-styrene sulfonic acid, 2-methyl-2-acrylamide propane sulfonic acid, methacryl acid phosphoxy propyl, methacryl 3-chloro-2-acid phosphoxy propyl, methacryl acid phosphoxy ethyl, itaconic acid, maleic acid, maleic anhydride, monoalkyl itaconate (e.g. methyl, ethyl, butyl, 2-ethyl hexyl), monalkyl maleate (e.g. methyl, ethyl, butyl, 2-ethyl hexyl; half-ester of acid anhydride with hydroxyl containing polymerisable unsaturated monomer (e.g. half-ester of succinic anhydride, maleic anhydride or phthalic anhydride with 2-hydroxy ethyl (meth)acrylate.

With respect to the monovalent organic acid (b), any aliphatic, aromatic, alicyclic or heterocyclic organic acids may be used. Typical examples of such acids are: acetic acid, propionic acid, levulinic acid benzoic acid, salicylic acid, lactic acid, 3,5-dichlorobenzoic acid, lauric acid, stearic acid, nitrobenzoic acid, linolenic acid, ricinoleic acid, 12-hydroxy stearic acid, fluoroacetic acid, pulvinic acid, abietic acid, mercaptobenzothiazole, o-cresotic acid, naphthol-1-carboxylic acid, p-phenyl benzene sulfonic acid, p-oxybenzoic acid, chloroacetic acid, dichloroacetic acid, naphthenic acid, b-naphthalene sulphonic acid, naphthol-1-sulfonic acid, 5-chloro-α,α-bis(3,5-dichloro-2-hydroxyphenyl)toluene sulphonic acid, p-phenyl benzoic acid, p-toluene sulphonic acid, p-benzene chlorosulphonic acid, dimethyl dithio carbamic acid, diethyl dithio carbamic acid, dibutyl dithiocarbamic acid, lithocholic acid, phenoxy acetic acid, 2,4-dichlorophenoxy acetic acid, pivalic acid, valeric acid and various synthetic fatty acids.

With respect to the above-mentioned other polymerisable unsaturated monomers, any customarily used ethylenically unsaturated monomer may be used. Examples of such monomers are methyl acrylate, methyl methacrylate, ethyl acrylate, ethyl methacrylate, propyl acrylate, propyl methacrylate, butyl acrylate, butyl methacrylate, octyl acrylate, octyl methacrylate, 2-ethyl hexyl acrylate, 2-ethyl hexyl methacrylate, methoxy ethyl methacrylate, styrene, vinyl toluene, vinyl pyridine, vinyl pyrolidone, vinyl acetate, acrylonitrile, methacrylo nitrile, dimethyl itaconate, dibutyl itaconate, di-2-ethyl hexyl itaconate, dimethyl maleate, di (2-ethyl hexyl) maleate, ethylene, propylene and vinyl chloride. One particular type of co-monomers is acrylic or methacrylic esters wherein the alcohol residue includes a bulky hydrocarbon radical or a soft segment, for example a branched alkyl ester having 4 or more carbon atoms or a cycloalkyl ester having 6 or more atoms, a polyalkylene glycol monoacrylate or monomethacrylate optionally having a terminal alkyl ether group or an adduct of 2-hydroxyethyl acrylate or methacrylate with caprolactone, e.g. as described in EP 0 779 304 A1”

If desired, hydroxy-containing monomers, such as 2-hydroxy ethyl acrylate, 2-hydroxy ethyl methacrylate, 2-hydroxy propyl acrylate, 2-hydroxy propyl methacrylate may also be used.

With respect to the polymers (d) and (g) which have an organic acid group at the side chain, mention is made of organic acids bearing vinyl resins, polyester resins, oil modified alkyd resins, fatty acid modified alkyd resins and/or epoxy resins.

It should be noted that in the resulting co-polymer, not all the organic acid side groups need to contain a metal ester bond; some of the organic acid side groups may be left un-reacted in the form of free acid, if desired.

The weight average molecular weight of the metal-containing co-polymer is generally in the range of from 1,000 to 150,000, such as in the range of from 3,000 to 100,000, preferably in the range of from 5,000 to 60,000.

Although a number of different methods for determining the weight average molecular weight of the polymer in question will be known to the person skilled in the art, it is preferred that the weight average molecular weight is determined in accordance with the GPC-method described at page 34 in WO 97/44401.

In another interesting embodiment of the invention the coating composition further comprises an amount of an organic ligand at least equal to the ligand-to-metal co-ordination ratio of 1:1, said organic ligand being selected from the group consisting of aromatic nitro compounds, nitriles, urea compounds, alcohols, phenols, aldehydes, ketones, carboxylic acids and organic sulphur compounds, whereby the co-polymer defined above forms a polymer complex with the organic ligand in situ.

Thus, if the above-defined co-polymer is considered as a hybrid salt then, by co-ordinating an organic ligand to each metal atom, the ion-association of the hybrid salt is retarded significantly to have a lower viscosity in a solution compared to the corresponding solution not containing the organic ligand. Furthermore, improvements may be found both in the sustained release of metal ions and the film consumption rate. Another important advantage is the fact that the complex hybrid salt is no longer reactive with conventional antifouling agents and pigments such as cuprous oxide, zinc oxide and the like. Therefore, the coating composition of the present invention is compatible with the conventional antifouling agents and pigments.

Examples of monobasic organic acids usable for forming the hybrid salt include monocarboxylic acids such as acetic, propionic, butyric, lauric, stearic, linolic, oleic, naphthenic, chloroacetic fluoroacetic, abietic, phenoxyacetic, valeric, dichlorophenoxy-acetic, benzoic or napthoic acid; and monosulphonic acids such as benzenesulphonic acid, p-toluenesulphonic acid, dodecylbenzenesulphonic acid, naphthalenesulphonic or p-phenylbenzenesulforic acid.

A preferred method for producing the polymeric hybrid salt has been disclosed in Japanese Patent Kokai No. 16809/1989. According to this method, copolymers containing pendant acid groups are reacted with a metal salt of a low boiling point-monobasic organic acid and a high boiling point-monobasic organic acid simultaneously to form a hybrid salt in which both the polymer pendant acid anion and the high boiling point-monobasic acid anion are bound to the same metal cation. For example, a hybrid copper salt with the polymeric acid and naphthenic acid may be obtained by reacting the polymeric acid with cupric acetate and naphthenic acid.

The polymer hybrid salts thus produced take a pseudo-cross-linked form due to ion-association and, therefore, have a relatively high viscosity in solutions. However, the viscosity may be decreased significantly by co-ordinating a further ligand to the hybrid salt as described herein. The resulting polymer complex thus formed also exhibits a relatively constant rate both in metal release and film consumption when applied as an antifouling coating film.

Organic ligands used for this purpose are selected from the group consisting of aromatic nitro compounds, urea compounds, nitriles, alcohols, phenols, aldehydes, ketones, carboxylic acids, and organic sulphur compounds. The organic ligands are not limited to unidentate ligands but also include polydentate ligand containing a plurality of identical or different ligating atoms in the ligand molecule.

Specific examples of such ligands include aromatic nitro, compounds such as nitrobenzene; nitriles such as isophthalonitrile; urea compounds such as urea, thiourea, N-(3,4-dichlophenyl)-N′-methoxy-N′-methylurea or N-(3,4-dichlorophenyl)-N′,N′-dimethylurea; alcohols such as butanol, octanol or geraniol; phenols such as hydroquinone, hydroquinone monomethyl ether, nonylphenol or BHT; aldehydes such as acetaldehyde or propionaldehyde; ketones such as acetylacetone, acetophenone or 2-amino-3-chloro-1,4-naphthoquine; carboxylic acids such as acetic acid, propionic acid, benzoic acid, lactic acid, malic acid, citric acid, tartaric acid or glycine; and sulphur compounds such as thiophene and its derivatives, n-propyl p-toluenesulphonate, mercaptobenzothiazole, dimethyldithiocarbamate or benzeneisothiocyanate. Some of these ligands may be used for antifouling purposes in conventional antifouling coating compositions.

The amount of organic ligand for complexing the polymer hybrid salt should be at least equal to the ligand-to-metal co-ordination ratio of 1:1. The maximum will be such an amount to saturate the co-ordination number of a particular metal used. For example, when a metal species having a coordination number of 4 is used, one or two moles of unidentate ligands or one mole of bidentate ligand may be co-ordinated to the metal atom.

The organic ligands are incorporated to a solution or varnish of the polymer hybrid salt to form a polymer complex in situ. The presence of excessive amounts of the organic ligands may be tolerated unless coating films are adversely affected such as occurrence of cracks or blisters when soaked in saline. The complexed copolymer may have a metal content from 0.3 to 20%, preferably from 0.5 to 15% by weight.

Examples of such further binder components are: oils such as linseed oil and derivatives thereof, castor oil and derivatives thereof, soy bean oil and derivatives thereof; and other polymeric binder components such as saturated polyester resins; polyvinylacetate, polyvinylbutyrate, polyvinylchloride-acetate, copolymers of vinyl acetate and vinyl isobutyl ether; vinylchloride; copolymers of vinyl chloride and vinyl isobutyl ether; alkyd resins or modified alkyd resins; hydrocarbon resins such as petroleum fraction condensates; chlorinated polyolefines such as chlorinated rubber, chlorinated polyethylene, chlorinated polypropylene; styrene copolymers such as styrene/butadiene copolymers, styrene/methacrylate and styrene/acrylate copolymers; acrylic resins such as homopolymers and copolymers of methyl methacrylate, ethyl methacrylate, n-butyl methacrylate, isobutyl methacrylate and isobutyl methacrylate; hydroxy-acrylate copolymers; polyamide resins such as polyamide based on dimerised fatty acids, such as dimerised tall oil fatty acids; cyclised rubbers; epoxy esters; epoxy urethanes; polyurethanes; epoxy polymers; hydroxy-polyether resins; polyamine resins; etc., as well as copolymers thereof.

It should be understood that the group of other polymeric binder components may include polymeric flexibilisers such as those generally and specifically defined in WO 97/44401 that is hereby incorporated by reference.

The dry matter of such further binder components is typically 0-10% by wet weight.

### Further Components/Aspects

The compositions of the present invention may be formulated as coatings, lacquers, stains, enamels and the like, hereinafter referred to generically as “coating(s)”.

Thus, in one aspect the present invention provides a coating consisting of a composition as defined herein.

Preferably, the coating is formulated for treatment of any surface that is in contact with water ranging from occasional humidity to constant immersion in water and which thus has the potential to be fouled. More preferably, the surface is selected from outdoor wood work, external surface of a central heating or cooling system, bathroom walls, hull of a marine vessel or any off-shore installations, and surfaces in food production/packaging and/or any other industrial processes.

The coating may include a liquid vehicle (solvent) for dissolving or suspending the composition.

The liquid vehicle may be selected from any liquid which does not interfere with the activities of any essential components of the composition. In particular, the liquid vehicle should not interfere with the activity of the essential enzyme(s) and/or anti-foulant compound. Suitable liquid vehicles are disclosed in U.S. Pat. No. 5,071,479 and include water and organic solvents including aliphatic hydrocarbons, aromatic hydrocarbons, such as xylene, toluene, mixtures of aliphatic and aromatic hydrocarbons having boiling points between 100 and 320° C., preferably between 150 and 230° C.; high aromatic petroleum distillates, e.g., solvent naptha, distilled tar oil and mixtures thereof; alcohols such as butanol, octanol and glycols; vegetable and mineral oils; ketones such as acetone; petroleum fractions such as mineral spirits and kerosene, chlorinated hydrocarbons, glycol esters, glycol ester ethers, derivatives and mixtures thereof.

The liquid vehicle may contain at least one polar solvent, such as water, in admixture with an oily or oil-like low-volatility organic solvent, such as the mixture of aromatic and aliphatic solvents found in white spirits, also commonly called mineral spirits.

The vehicle may typically contain at least one of a diluent, an emulsifier, a wetting agent, a dispersing agent or other surface active agent. Examples of suitable emulsifiers are disclosed in U.S. Pat. No. 5,071,479 and include nonylphenol-ethylene oxide ethers, polyoxyethylene sorbitol esters or polyoxyethylene sorbitan esters of fatty acids, derivatives and mixtures thereof.

Any suitable surface coating material may be incorporated in the composition and/or coating of the present invention.

In one aspect, the coating may comprise acrylic resins and methacrylate resins. Suitably, the coating may comprise methyl methacrylate, n-butyl methacrylate, methacrylic acid terpolymer and poly(vinyl methyl ether).

The composition and/or coating of the present invention may contain pigments selected from inorganic pigments, such as titanium dioxide, ferric oxide, silica, talc, or china clay, organic pigments such as carbon black or dyes insoluble in sea water, derivatives and mixtures thereof.

The composition and/or coating of the present invention may contain materials such as rosin to provide controlled release of the anti-foulant compound, rosin being to a very slight extent soluble in sea water. Suitably the rosin may be present in a liquid vehicle as described herein, for example in a liquid vehicle comprising xylene.

The composition and/or coating of the present invention may contain plasticisers, rheology characteristic modifiers, other conventional ingredients and mixtures thereof.

The composition and/or coating of the present invention, particularly the coating, may further comprise an adjuvant conventionally employed in compositions used for protecting materials exposed to an aquatic environment. These adjuvants may be selected from additional fungicides or biocides, auxiliary solvents, processing additives such as defoamers, fixatives, plasticisers, UV-stabilizers or stability enhancers, water soluble or water insoluble dyes, color pigments, siccatives, corrosion inhibitors, thickeners or anti-settlement agents such as carboxymethyl cellulose, polyacrylic acid or polymethacrylic acid, anti-skinning agents, derivatives and mixtures thereof.

The additional fungicide(s) or biocide(s) used in the composition and/or coating of the present invention is preferably soluble in the liquid vehicle.

The anti-fouling composition may also comprise one or more biocides as is customary within the field. Examples of biocides are: metallo-dithiocarbamates such as bis(dimethyldithiocarbamato)zinc, ethylene-bis(dithiocarbamato)zinc, ethylene-bis-(dithiocarbamato) manganese, and complexes between these; bis(1-hydroxy-2(1H)-pyridinethionato-O,S)-copper; copper acrylate; bis(1-hydroxy-2(1H)-pyridinethionato-O,S)-zinc; phenyl(bispyridyl)-bismuth dichloride; metal biocides such as copper, copper metal alloys such as copper-nickel alloys; metal salts such as cuprous thiocyanate, basic copper carbonate, copper hydroxide, barium metaborate, and copper sulphide; copper compounds such as cuprous oxide; heterocyclic nitrogen compounds such as 3a,4,7,7a-tetrahydro-2-((trichloromethyl)-thio)-1H-isoindole-1,3(2H)-dione, pyridine-triphenylborane, 1-(2,4,6-trichlorophenyl)-1H-pyrrole-2,5-dione, 2,3,5,6-tetrachloro-4-(methylsulfonyl)-pyridine, 2-methylthio-4-tert-butylamino-6-cyclopropylamine-s-triazin, and quinoline derivatives; heterocyclic sulfur compounds such as 2-(4-thiazolyl)-benzimidazole, 4,5-dichloro-2-n-octyl-4-isothiazolin-3-one, 4,5-dichloro-2-octyl-3(2H)-isothiazoline, 1,2-benzisothiazolin-3-one, and 2-(thiocyanatomethylthio)-benzothiazole; urea derivatives such as N-(1,3-bis(hydroxymethyl)-2,5-dioxo-4-imidazolidinyl)-N,N′-bis(hydroxymethyl)urea, and N-(3,4-dichlorophenyl)-N,N-dimethylurea, N,N-dimethyl-chlorophenylurea; amides or imides of carboxylic acids; sulfonic acids and of sulfenic acids such as 2,4,6-trichlorophenyl maleimide, 1,1-dichloro-N-((dimethylamino)-sulfonyl)-1-fluoro-N-(4-methylphenyl)-methanesulfenamide, 2,2-dibromo-3-nitrilo-propionamide, N-(fluorodichloromethylthio)-phthalimide, N,N-dimethyl-N′-phenyl-N′-(fluorodichloromethylthio)-sulfamide, and N-methylol formamide; salts or esters of carboxylic acids such as 2-((3-iodo-2-propynyl)oxy)-ethanol phenylcarbamate and N,N-didecyl-N-methyl-poly(oxyethyl)ammonium propionate; amines such as dehydroabiethylamines and cocodimethylamine; substituted methane such as di(2-hydroxy-ethoxy)methane, 5,5′-dichloro-2,2′-dihydroxydiphenylmethane, and methylene-bisthiocyanate; substituted benzene such as 2,4,5,6-tetrachloro-1,3-benzenedicarbonitrile, 1,1-dichloro-N-((dimethylamino)-sulfonyl)-1-fluoro-N-phenylmethanesulfenamide, and 1-((diiodomethyl)sulfonyl)-4-methyl-benzene; tetra-alkyl phosphonium halogenides such as tri-n-butyltetradecyl phosphonium chloride; guanidine derivatives such as n-dodecylguanidine hydrochloride; disulfides such as bis-(dimethylthiocarbamoyl)-disulfide, tetramethylthiuram disulfide; imidazole containing compound, such as medetomidine; 2-(p-chlorophenyl)-3-cyano-4-bromo-5-trifluoromethylpyrrole and mixtures thereof.

In one aspect the present invention provides a marine anti-foulant consisting of a composition as defined herein.

Preferably, the anti-foulant is self-polishing.

When used herein, the term “self-polishing” is intended to mean that the paint coat (i.e. the dried film of the coating composition) should have a polishing rate of at least 1 per 10,000 Nautical miles (18,520 km), determined in accordance with the “Polishing rate test” specified below. Preferably the polishing rate is in the range of 1-50 μm, in particular in the range of 1-30 μm per 10,000 Nautical miles (18,520 km).

### Polishing Rate Test

Polishing and leaching characteristics are measured using a rotary set-up similar to the one described by Kiil et al. (Kiil, S, Weinell, C E, Yebra, D M, Dam-Johansen, K, “Marine biofouling protection: design of controlled release antifouling paints.” In: Ng, K M, Gani, R, Dam-Johansen, K (ed) Chemical Product Design; Towards a Perspective Through Case Studies, 231 DBN-13: 978-0-444-52217-7. Part II (7), Elsevier. (2006)). The set-up consists of a rotary rig, which has two concentric cylinders with the inner cylinder (rotor, diameter of 0.3 m and height 0.17 m) capable of rotation. The cylinder pair is immersed in a tank containing about 400-500 litres of artificial seawater (32 g/l NaCl, 14 g/l MgSO₄.7H₂O and 0.2 g/l NaHCO₃).

The tank is fitted with baffles to break the liquid flow, which enhances turbulence and enables faster mixing of the species released from the paints and enhance heat transfer from a thermostating system. The purpose of using two cylinders is to create a close approximation to couette flow (flow between two parallel walls, where one wall moves at a constant velocity). The rotor is operated at 20 knots and the pH is adjusted frequently to 8.2 using 1 M sodium hydroxide or 1 M hydrochloric acid.

Samples are prepared using overhead transparencies (3M PP2410) that are primed using two-component paint (Hempadur 4518 ex Hempel's Marine Paints A/S) applied using a Doctor Blade applicator with a gap size of 200 μm. Coating samples are applied adjacent to each other using a Doctor Blade applicator with a gap of 250 μm. After drying for 1 day, the coated transparency is cut in strips of 2 cm resulting in eight samples of 1.5×2 cm² on a long (21 cm) strip. The strips are mounted on the rotor, and left to dry for a week.

After one week, the test is initiated, and during the experiment, samples are removed after 35, 65 and 140 days in order to inspect the polishing and leaching depths. The samples are dried for three days at ambient conditions, after which they are cut in half and cast in paraffin. The internal front of the sample is planed off before total film thickness and leached layer thickness is established using light microscopy (coating cross-section inspection).

The composition of the present invention can be provided as a ready-for-use product or as a concentrate. The ready-for-use product may be in the form of a powder, an oil solution, oil dispersion, emulsion, or an aerosol preparation. The concentrate can be used, for example, as an additive for coating, or can be diluted prior to use with additional solvents or suspending agents.

An aerosol preparation according to the invention may be obtained in the usual manner by incorporating the composition of the present invention comprising or suspended in, a suitable solvent, in a volatile liquid suitable for use as a propellant, for example the mixture of chlorine and fluorine derivatives of methane and ethane commercially available under the trademark “Freon”, or compressed air.

As discussed in U.S. Pat. No. 5,071,479 the composition and/or coating of the present invention may include additional ingredients known to be useful in preservatives and/or coatings. Such ingredients include fixatives such as carboxymethylcellulose, polyvinyl alcohol, paraffin, co-solvents, such as ethylglycol acetate and methoxypropyl acetate, plasticisers such as benzoic acid esters and phthlates, e.g., dibutyl phthalate, dioctyl phthalate and didodecyl phthalate, derivatives and mixtures thereof. Optionally dyes, color pigments, corrosion inhibitors, chemical stabilizers or siccatives (dryers) such as cobalt octate and cobalt naphthenate, may also be included depending on specific applications.

The composition and/or coating of the present invention can be applied by any of the techniques known in the art including brushing, spraying, roll coating, dipping and combinations thereof.

Compositions of the present invention can be prepared simply by mixing the various ingredients at a temperature at which they are not adversely affected. Preparation conditions are not critical. Equipment and methods conventionally employed in the manufacture of coating and similar compositions can be advantageously employed.

The invention will now be described in further detail by way of example only with reference to the accompanying figures in which:

FIG. 1 shows the enzyme based antifouling system.

FIG. 2 shows the HOX activity in varying concentrations of PEI (polyethyleneimine).

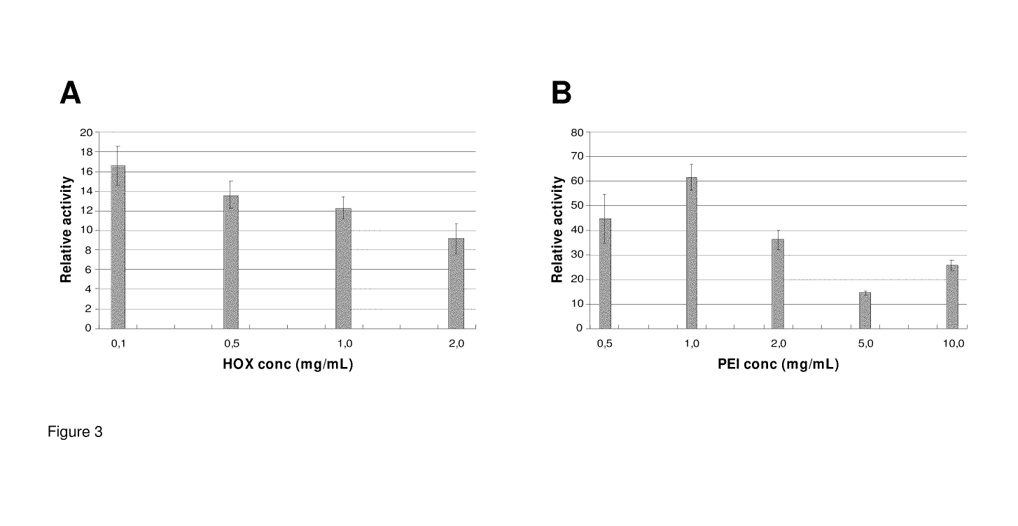

FIG. 3 shows the silica retained HOX activity with varying HOX and PEI concentrations.

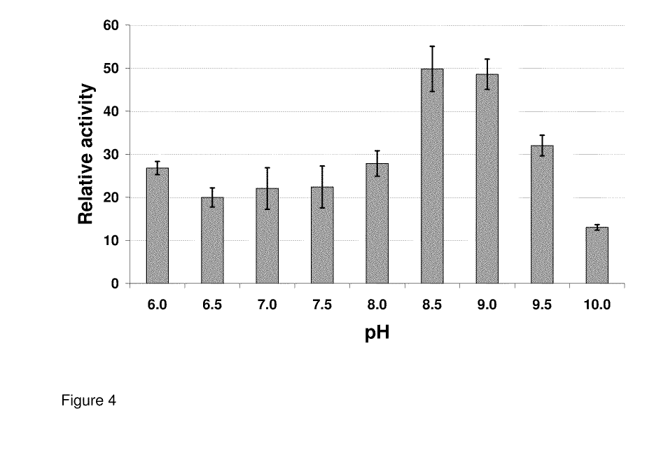

FIG. 4 shows the silica retained HOX activity with varying pH. The pH of the PBSi solution was varied as indicated in the graph. The highest activity is obtained at a pH of 8.5 to 9.

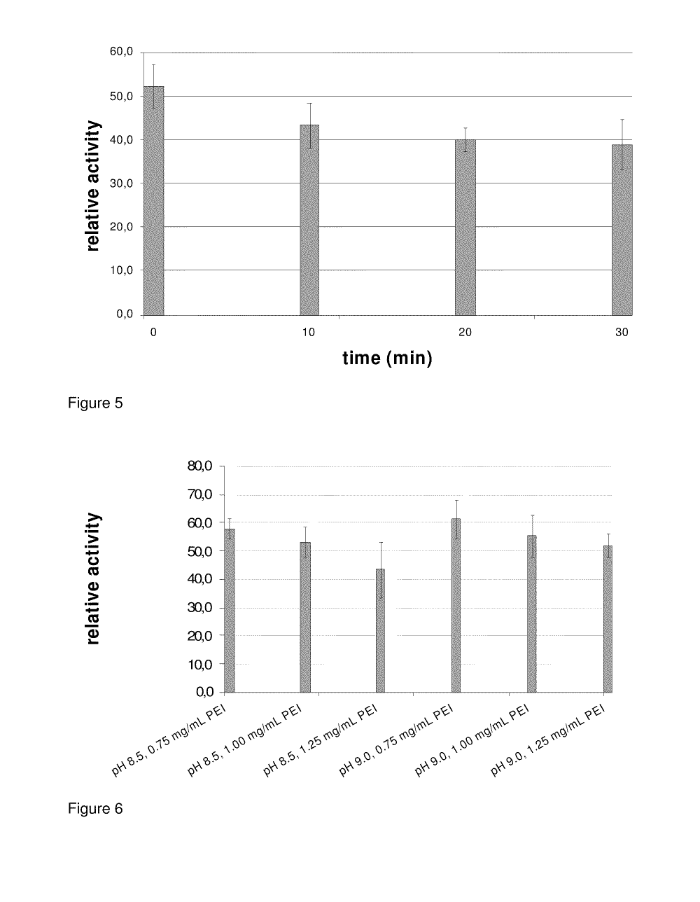

FIG. 5 shows the time scale HOX:PEI incubation.

FIG. 6 shows the second round of HOX silicate co-precipitation optimization.

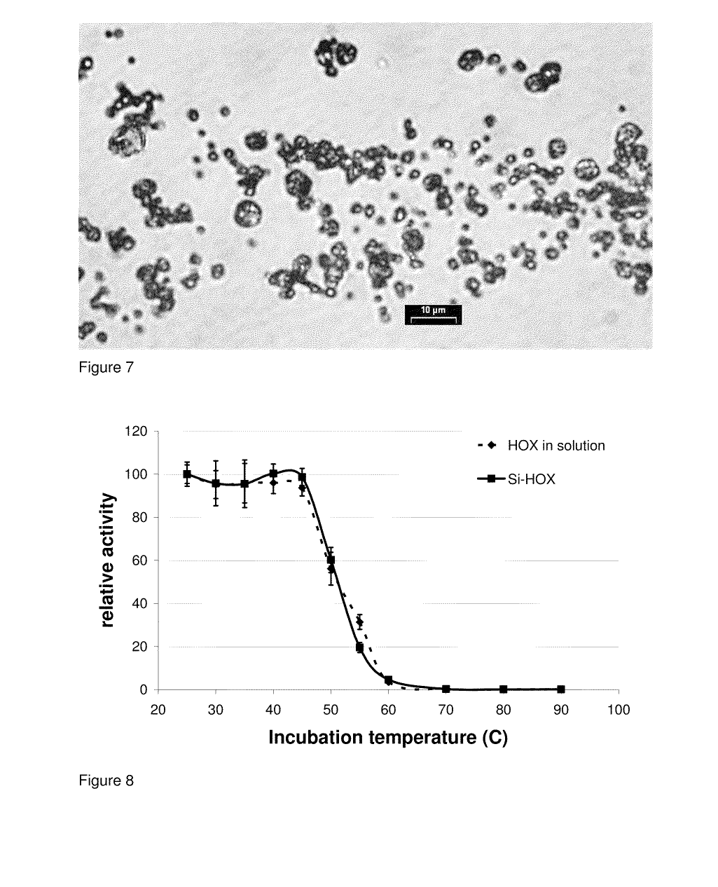

FIG. 7 shows the silica-HOX powder at 600× magnification.

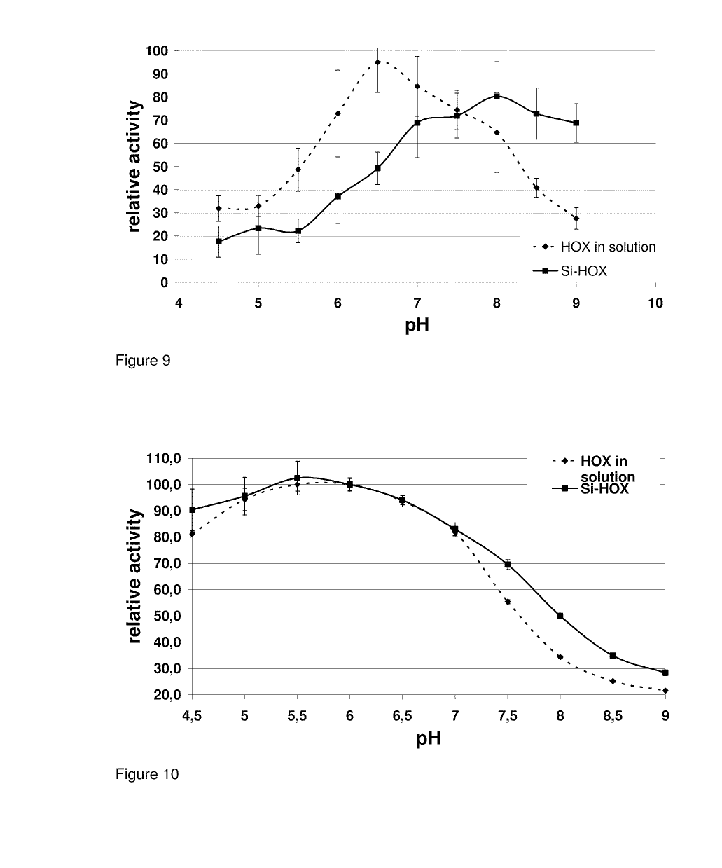

FIG. 9 shows the enzyme pH stability.

FIG. 10 shows the enzyme pH dependent activity.

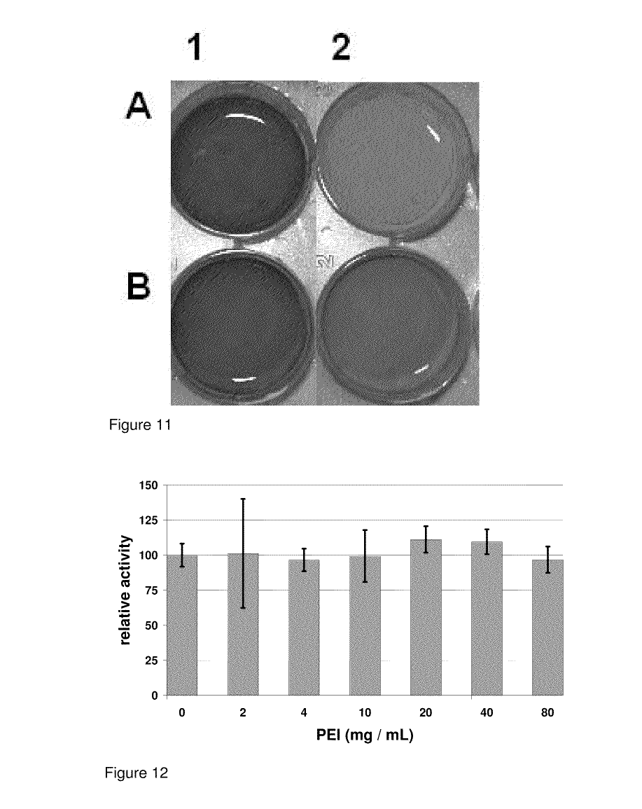

FIG. 11 shows the in-paint activity of Silica-HOX and free HOX.

FIG. 12 shows the GOX compatibility with PEI. The activity of GOX without PEI addition was set to 100 and the activity of the remaining samples set relative to that.

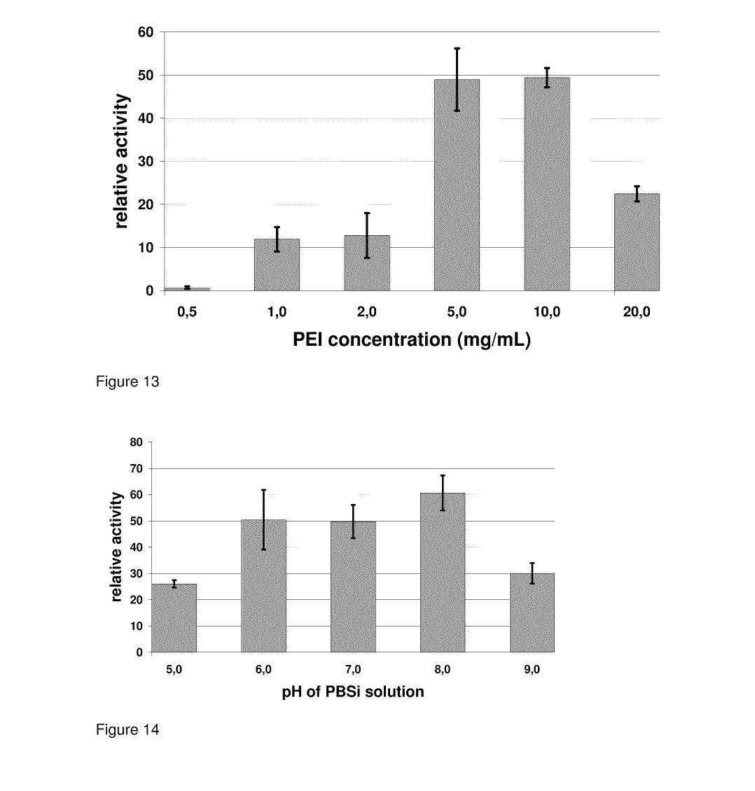

FIG. 13 shows the optimisation of PEI concentration in the GOX co-precipitation reaction. The activity of the free enzyme was set to 100 and the activity of the co-precipitates is given relative to that.

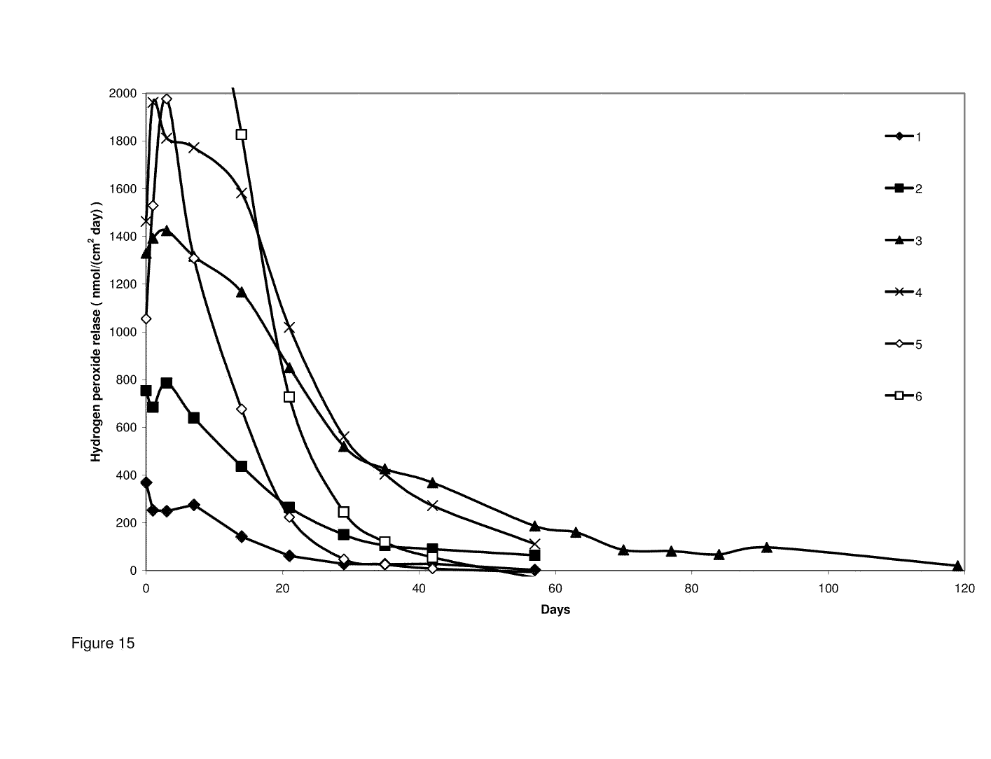

FIG. 15 shows the release of H₂O₂ from a surface painted with the samples described in table 4.

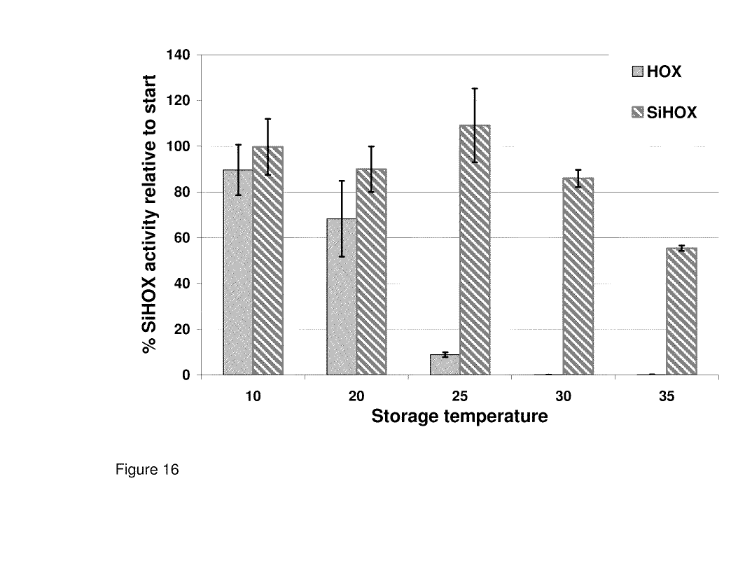

FIG. 16 shows the long term storage stability of HOX and SiHOX at different temperatures.

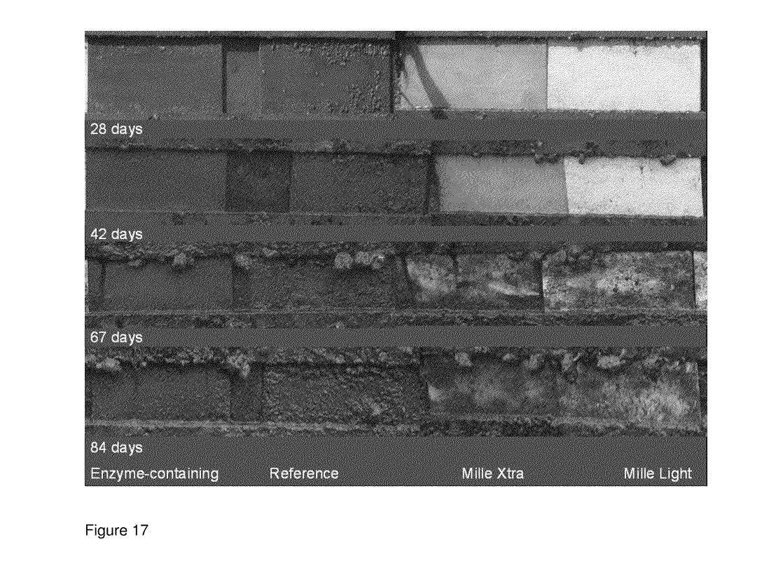

FIG. 17 shows the panels in the field raft trial in the North Sea, after 28 days, 42 days, 67 days, and 84 days. The panels are coated with (from left): H₂O₂-releasing enzyme-containing coating; the corresponding enzyme-free reference coating; Cu2O-based Hempel Mille Xtra; biocide-free Hempel Mille Light.

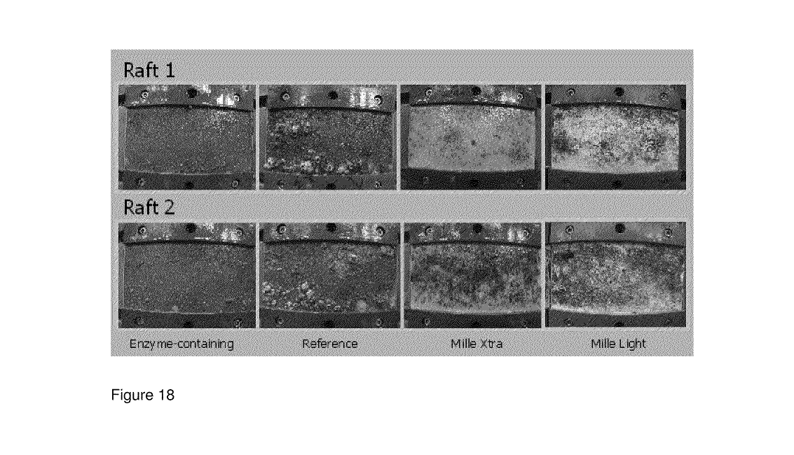

FIG. 18 shows the panels from the field raft trial in the North Sea, after 97 days.

The invention will now be described, by way of example only, in the following Examples.

### EXAMPLES

The anti-fouling effect of an anti-fouling composition of the present invention is tested according to the following examples. These Examples show the effectiveness of the present composition at preventing fouling. The Examples also provide for the optimisation of the anti-fouling properties of the present composition.

### Silicate-Coprecipitation

### Materials:

Sodium silicate solution: Na₂O₇Si₃ (27% SiO₂, 10% NaOH), (Sigma-Aldrich 13729-1L)
Polyethyleneimine (PEI): 50% w/v in H₂O, Mₙ 60 kDa, (Sigma-Aldrich P3143) Phosphoric acid, 85%
HOX: Hexose oxidase from Chondrus crispus was used as fermentation broth prepared as described in EP-A-0832245
GOX: Glucose oxidase GC199 available from Genencor International Inc, Rochester, N.Y., USA.
GA: Glucoamylase from Trichoderma reesei was used as fermentation broth prepared as described in US 2006/0094080

### General Procedure:

1. Mix enzyme (1-100 mg/mL) with Polyethyleneimine (2-20% w/v) so that Polyethyleneimine (PEI) is present in 1 to 5 fold excess by weight.

2. Add to freshly prepared phosphate-buffered silicate solution (5 to 10 fold excess v/v pH 5-8, 100-200 mM SiO₂) (prepared from sodium silicate solution and to phosphoric acid)

3. Mix 30 min

4. Collect solid by centrifugation or alternatively spray dry the product

Exact parameters used are described in the results section.

### Activity Measurements

### HOX/GOX:

The HOX or GOX activity was measured according to Savary et al., 2001, Enzyme and Microbial Technology; 29:42-51). One unit is defined as the amount of enzyme required to produce 1 μmol H₂O₂ pr minute at 25° C. at the given conditions. The plate reader was set to mix in-between each measurement in order to ensure a homogenous distribution of the silica-HOX/GOX particles. The dilute suspension of particles did not interfere with the assay.

### Glucoamylase:

GA activity was measured using a megazyme assay (R-AMGR3 05/04) based on p-nitrophenyl-β-maltoside and β-glucosidase according to manufacturers instructions.

Artificial sea water: NaCl: 24.0 g/L, MgCl₂ 5.1 g/L, Na₂SO₄ 4.0 g/L, CaCl₂ 1.1 g/L, KCl 0.67 g/L, KBr 0.098 g/L, H₃BO₃ 0.027 g/L, SrCl₂ 0.024 g/L, NaF 0.003 g/L, NaHCO₃ 0.196 g/L.

### Results

### Example 1

### Activity of HOX in PEI

The activity of HOX in varying concentrations of PEI was determined to investigate possible inhibition. HOX (500 μL, 1.5 mg) was mixed with 0, 1.5, 3, 10, 20 and 30 mg PEI in 600 μL water. The solution was allowed to incubate for 10 min at RT and HOX activity measured. All samples were prepared and assayed in duplicate.

The activity of HOX in the presence of varying concentrations of PEI relative to the activity without PEI is shown in FIG. 2.

The results clearly indicate some inhibition of HOX in high concentrations of PEI, only 70% activity is retained when using 20 fold excess (30 mg) of PEI.

### Example 2

### HOX-Silicate Co-Precipitation

A freshly prepared solution (100 μL) of HOX (1 mg/mL) and PEI (1, 5 or 10 mg/mL) was mixed into 400 μL of a fresh solution of 100 mM PBSi and mixed for 10 min. The samples were centrifuged for 2 min at 800 g and the resulting pellets were washed twice in 500 μL H₂O and resuspended in 1 mL H₂O. HOX activity was measured in the HOX:PEI solutions, the resuspended pellets and the supernatants from the precipitation reaction (see table 1):

TABLE 1
Relative HOX activity in different fractions
from co-precipitation reaction.

| | PEI | Activity in | Activity in |
|---|---|---|---|
| | concentration | reaction | silica |
| Sample# | (mg/mL) | supernatant | Pellet |
| 1 | 1 | 1% | 27% |
| 2 | 5 | 0% | 62% |
| 3 | 10 | 21% | 57% |

The indicated activity is in percent of the activity of total enzyme added. The experiments were performed in duplicate and the average activity recorded

It can be concluded that HOX can be encapsulated using silicate co-precipitation.

### Example 3

### Optimization of HOX-Silicate Co-Precipitation

A series of HOX-silicate co-precipitations were performed to optimize the HOX activity in the silica particles. The reactions were carried out as described in example 2 using the following conditions except for the varied parameter described in each figure: 100 mM PBSi, pH 6.5; 1 mg/mL HOX in HOX:PEI solution; 5 mg/mL PEI in HOX:PEI solution; Reactions were performed at room temperature.

The precipitation reactions were performed in triplicate and activity assays in duplicate for each sample and the recorded activity is given in percentage of activity of the same amount of enzyme in solution. The results are summarised in FIGS. 3-5.

FIG. 3A: Four different concentrations of HOX were tested as indicated. The graphs show the relative activity with respect to the amount of enzyme added. Therefore, please note that the specific activity of the silica material with higher concentrations of HOX is higher than with lower concentrations of HOX, but the relative activity loss is lowest with lowest enzyme concentrations.

FIG. 3B: Five different concentrations of PEI were tested as indicated. A cloudy precipitate was observed in the sample with 5 mg/mL PEI but not the sample with 10 mg/mL

The incubation time of HOX:PEI before the solution was added to PBSi was varied as indicated in the graph. Some activity is lost during longer incubation times.

In summary, the best activity was obtained with shortest possible HOX:PEI incubation times prior to addition to the PBSi solution. Low concentration of both enzyme and PEI gave best relative activity. The optimal pH of the PBSi was shown to be around 9. Using low concentrations of enzyme (1 mg/mL), the captured activity was unchanged using PBSi concentrations between 50 and 200 mM (not shown).

It was decided not to go lower in enzyme concentration than 1 mg/mL because even though the relative loss in enzyme activity will be lower using lower enzyme concentrations, the absolute activity of the silica particles will be too low. A second round of optimization was performed, varying PEI concentration and pH value near the found optimum values (FIG. 6).

PEI concentration and pH was optimized around previously found optimum values. The best activity is found at pH 9 with a PEI concentration of 0.75 mg/mL.

The highest retained activity was around 60%. However, the final yield in production facilities will be highly dependent on process equipment and parameters.

### Example 4

### Upscaling and Optimization of Spray-Drying Parameters

The process was scaled up to 2 L total volumes based on the best result from the activity optimization. In order to transfer the silica particles from the aqueous solution to xylene, spray-drying was employed.

Parameters:
100 mM PBSi, pH 9.0
1 mg/mL HOX in HOX:PEI solution
0.75 mg/mL PEI in HOX:PEI solution
1.6 L PBSi was mixed with 400 mL HOX:PEI for 30 min at 4° C.

Spray-drying was performed on a lab scale spray dryer (BÜCHI Mini Spray Dryer B-191) using an air flow of 600 L/h, and inlet temperature of 180° C. and outlet of 85° C. The aspirator was set to 80% and cooling was applied at spray nozzle.

The procedure resulted in a silica-HOX powder with an activity of 380 HOX U/g powder.

### Example 5

### Microscopy of Silica Encapsulated HOX

A picture of the dry powder at 600× magnification is shown in FIG. 7.

Particles as seen using light microscopy on the dry powder

Based on microscopy, the particle size has been measured to be 5 μm or below for most particles (FIG. 7), and the particle size does not appear to be affected by hydration of the particles (not shown). The size is thus suitable for paint.

### Example 6

### Comparison of Silica Encapsulated HOX (SiHOX) and Non-Encapsulated HOX

The enzyme temperature stability of HOX in solution and silica encapsulated HOX was compared. As can be seen in FIG. 8, there is no significant difference in temperature stability between the samples.

The relative activity of free HOX and silica encapsulated HOX after 10 min incubation at the given temperature (in 100 mM phosphate pH 6.3). The activity at 25° C. was set to 100. No significant difference in temperature stability was observed.

The pH stability of silica encapsulated HOX and HOX in solution was compared by incubating the two samples in 100 mM phosphate buffer with a given pH at RT for 24 h (FIG. 9). At pH above 7.5 the silica encapsulated HOX was more stable than HOX in solution.

The pH dependent stability of HOX in solution and HOX encapsulated in silica was compared by incubating at RT for 24 h in a 100 mM phosphate buffer at the indicated pH. The activity was assayed at standard conditions (25° C., pH 6.5). The activity of a sample stored 24 h at pH 6.5 and 4° C. was set to 100%.

The HOX activity at different pH values was measured using the standard HOX assay with the exception that the phosphate buffer was adjusted to the indicated pH value. The activity at pH 6.0 was set to 100%. Silica encapsulated HOX has higher activity than HOX in solution at pH above 7.

At pH 8, which is relevant to sea water (7.5-8.5), silica encapsulated HOX retained 50% activity compared to the activity at pH optimum (6.0), whereas HOX in solution only has 35% of the optimum activity. Moreover, the silica encapsulated HOX was more stable at pH relevant to sea water.

### Example 7

### In-Paint Activity of Silica-HOX

In order to test in-paint activity, silica-HOX and HOX on starch (HOX HP 1.0 K, Danisco: 1223416, 666 HOX/g) was added to a final concentration of 80 U pr g paint (Hempel Mille light 71400). Paint samples of 0.5 g were added to a 6 well polystyrene plate and allowed to dry for three days.

After drying, the wells were washed in water and enzyme activity was qualitatively verified by adding 2 mL of substrate to each well (FIG. 11 row 1). The surfaces were immersed in ASW for three days and enzyme activity qualitatively measured again (FIG. 11, row 2)

In-paint activity of Silica encapsulated HOX and free HOX were assayed as described in the text. A: SiHOX; B: free HOX. Row 1: Activity assay immediately after immersion in ASW. Row 2: activity after three days of immersion in ASW. The green colour developed within 2-3 minutes.

The in-paint activity immediately after hydration of the paint (FIG. 11, row 1) was comparable for HOX encapsulated in silica and HOX formulated on starch. However, after three days of immersion, the activity of the starch-HOX was significantly lower than the Si—HOX. The observed picture was the same after 20 days (not shown). As expected, the silica encapsulation provides a better retention of the enzyme activity in the paint.

### Example 8

### Preparation of Silica-GOX

Silica co-precipitation was tested for glucose oxidase (GOX) in order to make sure that the system not only works for HOX. The same general procedure as described above for HOX was used.

GOX Compatibility with PEI

It was observed (example 1) that HOX is sensitive to addition of PEI and incubation time of HOX and PEI have a significant impact on the resulting enzyme activity of the co-precipitates. Enzyme activity of GOX after incubation in increasing amounts of PEI was determined in order to investigate if the same is the case for GOX. GOX (2 mg/mL in 100 mM phosphate buffer pH 6.3) was mixed with PEI to give a final volume of 1 mL with PEI concentration as indicated in FIG. 12.

Contrary to the results with HOX it can be seen that GOX activity is not sensitive to the presence of PEI.

### Optimisation of PEI Concentration in Co-Precipitation Reaction

Based on previous experience, the most important parameters for efficient capture of to enzyme activity in the formed silica particles are PEI concentration and pH. The parameters were varied and the results shown below.

For optimisation of PEI concentration 400 μL PBSi pH 6.5 was mixed with 100 enzyme-PEI mixture where the enzyme concentration was kept at 1 mg/mL and the PEI concentration varied as indicated in FIG. 13:

The optimal concentration of PEI seems to be 5-10 times excess on a w/w basis with respect to enzyme.

### Optimization of pH in Co-Precipitation Reaction

For optimisation of pH, 400 μL PBSi adjusted to the pH given in FIG. 3 was mixed with 100 μL enzyme-PEI mixture where the enzyme concentration was kept at 1 mg/mL and the PEI concentration at 5 mg/mL.

The optimal pH for the PBSi solution of the precipitation reaction is in the area of pH 6 to 9.

### Preparation of Dried Silica Co-Precipitated GOX (SiGOX)

A medium scale preparation of SiGOX was prepared as described above using the following procedure. 1600 mL PBSi (100 mM, pH 7.5) was mixed with 400 mL GOX PEI solution where the enzyme concentration was kept at 1 mg/mL and the PEI concentration at 5 mg/mL. After 30 min of mixing the pH was adjusted to 5, 7 or 9 as indicated in Table 2 and the product dried using a Büchi spraydryer B191 with inlet temperature at 180° C. and outlet temperature at 85° C. and airflow of 600 Uh.

TABLE 2
Activity of obtained SiGOX powder

| pH of silica solution | Act (u/g powder) |
|---|---|
| 5.0 | 238 ± 47 |
| 7.0 | 272 ± 42 |
| 9.0 | 97 ± 10 |

It is observed that the best activity is obtained when spraydrying a solution of neutral pH.

### Example 9

### Preparation of Starch-GA-SiHOX/SiGOX System

Starch-enzyme formulations for paint were prepared with varying concentrations of glucoamylase and SiHOX/SiGOX in order to verify that the hydrogen peroxide release can be controlled by enzyme dosage and for use in future field trials of enzyme based coatings.

Samples with the composition indicated in table 3 were prepared using the following procedure. Liquid formulation of SiHOX was prepared as described in example 4 and SiGOX using the procedure described above with the following conditions: 100 mM PBSi, pH 7.5, GOX concentration in Enz:PEI 2 mg/mL, PEI concentration in Enz:PEI 10 mg/mL. Cornstarch (Cargill C*gel 03401, Cedar Rapids, Iowa) was mixed into 500 mL of the co-precipitate and glucoamylase (62000 U/g) added immediately before spray drying. Spray drying was performed on a Mini Spray Dryer B-191 (Büchi Labortechnik, Flawil, CH) using an inlet temperature of 135° C. and outlet of 80° C. and an airflow of 600 L/h. The aspirator was held at 90%. Cooling with ice-cold water was applied at spray nozzle.

TABLE 3
starch/GA/(SiHOX/SiGOX) preparations for paints.

| Sample | Starch | co-precipitate | GA | GA | HOX | HOX/GOX | H₂O₂ release |
|---|---|---|---|---|---|---|---|
| ID | (g) | (mL) | (mL) | level | level | activity (U/g) | (mU/g) |
| 1 | 75 | 500 mL SiHOX | 0.012 | 1 | 1 | 142.0 ± 13.3 | 85.3 ± 8.9 |
| 2 | 75 | 500 mL SiHOX | 0.115 | 10 | 1 | 161.9 ± 8.3 | 381.7 ± 38.1 |
| 3 | 75 | 500 mL SiHOX | 0.575 | 50 | 1 | 234.8 ± 14.7 | 832.9 ± 100.8 |
| 4 | 75 | 500 mL SiHOX | 1.15 | 100 | 1 | 211.1 ± 17.8 | 1309.1 ± 127.5 |
| 5 | 75 | 500 mL SiHOX | 0.575 | 50 | ½ | 100.8 ± 7.1 | 840.1 ± 91.8 |
| 6 | 50 | 500 mL SiGOX | 0.231 | 50 | — | ND | ND |
| 7 | 50 | 500 mL SiGOX | 0 | 0 | — | 268.1 ± 11.2 | — |
| 8 | 75 | 150 mL buffer | 0.575 | 50 | 0 | | *)474.6 ± 9.6 |
| 9 | 75 | 500 mL SiHOX | 0 | 0 | 1 | 147.9 ± 7.2 | — |
| 10 | 75 | 500 mL SiHOX | 0 | 0 | ½ | 70.6 ± 5.2 | — |

The samples were prepared as described in the text using the amounts of starch and enzyme indicated. The columns GA and HOX level indicate the level of the respective enzyme where the concentration has been set to 1 for sample 1. Measured HOX/GOX activity was measured in the presence of glucose. H₂O₂ release indicates the activity given in mU with in-situ glucose production using the same assay without glucose added.) This value is theoretical glucose release (nmol/min/g) based on GA activity assay, rather than hydrogen peroxide release. ND: not determined.

It is observed that the HOX activity shows some batch to batch variation, but it is the in-situ generation of glucose that will be the rate limiting step for all samples (HOX/GOX activity higher than H₂O₂ release). The hydrogen peroxide release can be controlled by the amount of GA but a linear dependency is not observed.

### Example 10

### In-Paint Activity of Full System

In order to test in-paint activity, 2 g of samples 1-6 indicated in table 3 was added to 18 g xylene based paint (Hempel Mille light 71400) and 5 mL xylene. The paint samples were applied in 6 well polystyrene plates and allowed to dry for two days before applying a new layer of paint that was allowed to dry for three days. The plates were immersed in a large volume of ASW and release of hydrogen peroxide from the surface assayed. Hydrogen peroxide release from the paint surface was assayed 2 h after immersion and after one week of immersion in ASW (table 4).

TABLE 4
hydrogen peroxide release from paint surfaces.

| Sample | GA | HOX | H₂O₂ release After 2 h | H₂O₂ release Day 7 |
|---|---|---|---|---|
| ID | level | level | (nmol/(cm²*day)) | (nmol/(cm²*day)) |
| 1 | 1 | 1 | 368 | 275 |
| 2 | 10 | 1 | 754 | 640 |
| 3 | 50 | 1 | 1330 | 1317 |
| 4 | 100 | 1 | 1463 | 1772 |
| 5 | 50 | ½ | 1055 | 1308 |
| 6 | 50 | GOX | 3504 | 3034 |

Painted surfaces were immersed in ASW and hydrogen peroxide release from the surface assayed after 2 h and after 7 days.

It is observed that there is no significant loss of hydrogen peroxide release from the surfaces after one week of immersion. The release can be efficiently controlled by the glucoamylase level.

The hydrogen peroxide release from a coating immersed in ASW for 17 weeks is shown for sample 3 and 8 weeks for samples 1, 2, and 4-6 in FIG. 15:

It is observed that the release of sample 3 decrease from approximately 1400 nmol/(cm²*day) to 520 nmol/(cm²*day) after 29 days, 67 nmol/(cm²*day) after 84 days, and 20 nmol/(cm²*day) after 119 days (i.e. 17 weeks). It is important to note that the experimental work in this example was carried out in a laboratory system where the polishing of the paint will be very low or non-existing. It is expected that the release will reach a steady state in the final application when the polishing rate has reached equilibrium.

Previously, antifouling activity was reported for a hydrogen peroxide release rate of 36 nmol/(cm²*day) from a photocatalysed hydrogen peroxide releasing antifouling paint (Morris and Walsh, 2000, U.S. Pat. No. 6,063,849). Further, Nippon Paint Co. has detailed a hydrogen peroxide release rate with antifouling activity of 21 nmol/(cm2*day) (Hamade and Yamamori, 2000, U.S. Pat. No. 6,150,146).

It is concluded that in a system with no or only weak polishing, sample 3 will be able to produce an antifouling effect for 3-4 months. If this is combined with a carefully adjusted polishing rate of the coating the obtained antifouling effect is expected to be comparable to the lifetime of current state of the art coatings.

### Example 11

### Storage Stability of HOX and SiHOX in Artificial Sea Water (ASW)

To determine the long-term stability of HOX and SiHOX enzyme in solution, enzymes were diluted approximately 2000-fold in sterile filtered Artificial seawater (ASW) to give an initial activity of 250 HOX u/L and stored at 10, 20, 25, 30 and 35° C. for 12 weeks. The enzyme activity in the samples after storage for 12 weeks at the indicated temperatures is plotted in FIG. 16 as percentage of the initial activity in the sample. It is observed that SiHOX provides better stability results than HOX at all of the temperatures tested. It is also observed that SiHOX has a significant better stability than HOX at temperatures from 25° C. and above. At 30 and 35° C. there is no detectable activity in the HOX samples. This shows that even though no significant difference in short term temperature stability was observed (Example 6), the long-term stability of the enzyme in sea water is increased when encapsulated in silica.

### Example 12

### Raft Trial of Enzyme-Based Hydrogen Peroxide Releasing Coating

Raft trials were performed in order to test the in-sea performance of the system.

Xylene-based rosin self-polishing coatings were provided by Hempel A/S, Kongens Lyngby, Denmark.

The xylene-based rosin self-polishing coatings were formulated with a volume composition of solids of 36% zinc resinate, 18% methyl methacrylate/n-butyl methacrylate/methacrylic acid terpolymer (100/100/1 molar ratio. Purchased as Degalan LP 64/12 from Rohm GMBH) and 6% poly(vinyl methyl ether), 10% inert red iron oxide pigment (Cas nr: 1309-37-1), and 30% cornstarch, which had previously been spray with GA and SiHOX as described in Example 9 sample 3, yielding the enzyme-containing coating.

In addition to the enzyme-based coating an enzyme-free reference coating and two commercial coatings were included in the test. These were the Cu₂O-containing Mille Xtra and the biocide free Mille Light (Hempel NS, Kongens Lyngby, Denmark). The coatings were applied in duplicate to rotor panels and mounted on rafts that were immersed in the North Sea. The raft trial started in July and ended beginning of November, with an average seawater temperature reported to 17° C. throughout the trial period. The rafts were regularly inspected visually for the nature and density of biofouling, and images were taken for documentation.

Images taken during (FIG. 17) the raft trial illustrate the gradual build-up of fouling over a 97-day period. The H₂O₂ producing test-coating with enzymes (far-left) was compared to an enzyme-free reference coating (center left), and the two commercial coatings containing either Cu₂O and biocide (Mille Xtra, center-right) or biocide free (Mille Light, far-right).

Late-juvenile to adult barnacles already covered significant parts of the reference coating after 28 days of immersion, while the enzyme-containing coating and the commercial coatings remained relatively clean (FIG. 17, Table 5).

TABLE 5
Observations from visual inspection of coated
panels from sea raft trial, July-October.

| | Enzyme- | | | |
|---|---|---|---|---|
| Day of | containing | Reference | | |
| inspection | coating | coating | Mille Xtra | Mille Light |
| 28 | Some slime | 10-20% area | Slime | Slime |
| | Few juvenile | Late-juvenile | | |
| | barnacles | to adult | | |
| | | barnacles | | |
| 42 | Slime | Slime | Slime | Slime |
| | 3-10 adult | 25-30 | Some young | Some young |
| | barnacles | individual | barnacles | barnacles |
| | Some young | adult | 10% area | 5% area |
| | barnacles | barnacles | green algae | green algae |
| 67 | Slime | Slime | Slime | Slime |
| | 5 adult | 30 adult | Some young | Some young |
| | barnacles | barnacles | barnacles | barnacles |
| | Some young | 10-20 | 5 adult | 5-15 adult |
| | barnacles | tunicates | barnacles | barnacles |
| | 10% area | 15% area | 20-30% area | 5% area |
| | diatoms | diatoms | diatoms | green algae |
| | Appeared less | | | 20% diatoms |
| | fouled than | | | |
| | other three | | | |
| | coatings | | | |
| | overall | | | |
| 84 | Slime | Slime | Slime | Slime |
| | Adult | Very abundant | Algae | Algae |
| | barnacles | adult | No further | No further |
| | Algae | barnacles | characterisa- | characterisa- |
| | No further | No further | tion possible | tion possible |
| | characterisa- | characterisa- | | |
| | tion possible | tion possible | | |
| 97 (after | Slime | Slime | Slime | Slime |
| short rotor | 6-12 barnacles | 10% area | 15-50% area | 25-60% area |
| treatment) | 10% area | diatoms | by diatoms | diatoms |
| | diatoms on one | 40 barnacles | | 10 barnacles, |
| | of the | on one | | 10% area |
| | duplicates | duplicate | | green algae |
| | | 35 barnacles | | and one |
| | | and 15% area | | oyster on |
| | | tunicates on | | one duplicate |
| | | second | | |
| | | duplicate | | |

Duplicate panels were used.

After 42 days, fouling was still moderate with notably denser settlement of barnacles on the enzyme-free reference coating compared to the others.

Almost four weeks later, at day 67, barnacles were abundant on the reference coating, and algae and diatoms had increased on the commercial coatings. Fouling on the enzyme-containing coating, however, had only increased slightly from day 42, and a pronounced difference was seen between this and the other coatings.

After 84 days, visual assessment of the mounted panels had become difficult because of sludge. On day 97 the panels were de-mounting from the rafts, the panels were then mounted on a rotor and subjected to a 10 knots rotary speed for 10 minutes. This mild rotary shear (washed off the sludge but left the adhered biofouling on the panels. At this point, the difference between the enzyme-containing coating and the reference became very clear (FIG. 18). The panels shown in FIG. 18 are coated with (from left): H₂O₂-releasing enzyme-containing coating; the enzyme-free reference coating; Cu₂O-based Hempel Mille Xtra; biocide-free Hempel Mille Light. Whereas the reference was covered by large quantities of barnacles in addition to slime and diatoms, the enzyme-containing coating had only a few adhered barnacles along with slime and diatoms. Mille Xtra had more diatoms than the enzyme-containing coating, but no barnacles, whereas one of the Mille Light panels had both barnacles and an oyster attached.

Overall performance of the paints at day 97 was scored using method derived from the ASTM International norm D6990 where Fouling Resistance (FR) of the panels is scored by subtracting either the coverage (expressed as a percentage) or actual number of organisms counted on each panel (and slime as 1 subtraction point) from a value of 100. Values between 0 (no antifouling activity at all) and 100 (perfectly clean paint surface) indicate the degree of protection of the individual panels through the trial. The higher the FR rate, the better the antifouling performance. The enzyme-containing coating achieved the highest collective fouling resistance score (table 6), even better than Mille Xtra, and superior to the enzyme-free reference.

TABLE 6
Fouling resistance (FR) of coatings calculated
after 97 days of immersion.

| | FR of both | |
|---|---|---|
| | panels | FR (avg) |
| Enzyme containing coating | 93; 77 | 85 |
| Reference coating | 49; 39 | 44 |
| Mille Xtra | 84; 49 | 66.5 |
| Mille Light | 74; 18 | 46 |

All publications mentioned in the above specification are herein incorporated by reference. Various modifications and variations of the described methods and system of the invention will be apparent to those skilled in the art without departing from the to scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

1. An anti-fouling composition comprising
(i) a surface coating material;
(ii) (a) a first enzyme; and
(b) a first substrate, wherein action of the first enzyme on the first substrate provides a second substrate;
(iil) a second enzyme, wherein the second enzyme is encapsulated in a silicate, and wherein said second enzyme generates an anti-foulant compound when acting on to said second substrate.

2. A composition according to claim 1, wherein the first enzyme is encapsulated.

3. A composition according to claim 1, wherein the encapsulated second enzyme is a co-precipitate of an enzyme, a silicate and a N-containing organic template molecule.

4. A composition according to claim 3, wherein the silicate is obtained by neutralising an alkali metal silicate.

5. A composition according to claim 3, wherein the N-containing organic template molecule is a polyamine, a modified polyamine, polyethyleneimine, a polypeptide or a modified polypeptide.

6. A composition according to claim 5, wherein the enzyme:polyethyleneimine ratio is from about 0.3 to about 10.

7. A composition according to claim 1 wherein the first enzyme is glucoamylase.

8. A composition according to claim 1 wherein the first substrate is an oligomer or a polymer of said second substrate.

9. A composition according to claim 1 wherein the first substrate is starch.

10. A composition according to claim 1 wherein the second enzyme is an oxidase.

11. A composition according to claim 1 wherein the second enzyme is an oxidoreductase.

12. A composition according to claim 1 wherein the second enzyme is selected from hexose oxidase and glucose oxidase.

13. A composition according to claim 1 wherein the second enzyme is a hexose oxidase.

14. A composition according to claim 1 wherein the second enzyme is a glucose oxidase.

15. A composition according to claim 1 wherein the ratio of first enzyme:second enzyme is at least 1:2.

16. A composition according to claim 1 wherein the ratio of first enzyme:second enzyme is at least 1:10.

17. A composition according to claim 1 wherein the ratio of first enzyme:second enzyme is at least 1:50.

18. A composition according to claim 1 wherein the second substrate is a sugar.

19. A composition according to claim 18 wherein the sugar is glucose.

20. A composition according to claim 1 wherein the composition further comprises a binder to immobilise at least one of the constituents of the composition.

21. A composition according to claim 20 wherein the binder immobilises at least the second enzyme.

22. A composition according to claim 1 wherein the composition is formulated as a coating, a lacquer, a stain or an enamel.

23. A composition according to claim 1 wherein the surface coating material comprises components selected from polyvinyl chloride resins in a solvent based system, chlorinated rubbers in a solvent based system, acrylic resins and methacrylate resins in solvent based or aqueous systems, vinyl chloride-vinyl acetate copolymer systems as aqueous dispersions or solvent based systems, polyvinyl methyl ether, butadiene copolymers such as butadiene-styrene rubbers, butadiene-acrylonitrile rubbers, and butadiene-styrene-acrylonitrile rubbers, drying oils such as linseed oil, alkyd resins, asphalt, epoxy resins, urethane resins, polyester resins, phenolic resins, natural) rosin, rosin derivatives, disproportionated rosin, partly polymerised rosin, hydrogenated rosin, gum rosin, disproportionated gum rosin, non-aqueous dispersion binder systems, silylated acrylate binder systems, metal acrylate binder systems, derivatives and mixtures thereof.

24. A composition according to claim 1 wherein the surface coating material is a resin.

25. A process for the preparation of a silicate encapsulated enzyme comprising the steps of
(i) providing a fermentation broth containing an enzyme or an enzyme isolated from a fermentation broth without drying,
(ii) encapsulating the enzyme in a silicate.

26. A process according to claim 25 characterised by the features of claim 1.

27. A silicate encapsulated enzyme obtained or obtainable by a process according to claim 25.

28. An anti-fouling composition comprising
(i) a surface coating material;
(ii) (a) a first enzyme; and
(b) a first substrate, wherein action of the first enzyme on the first substrate provides a second substrate;
(iil) a second enzyme, wherein the second enzyme is a silicate encapsulated enzyme according to claim 27, and wherein said second enzyme generates an anti-foulant compound when acting on said second substrate.

29. A coating consisting of a composition according to claim 1.

30. A marine anti-foul consisting of a composition according to claim 1.

31. A marine anti-foul according to claim 30 wherein the anti-foulant is self-polishing.

32. A method for releasing an anti-fouling compound from a surface coating, which method comprises incorporating in a surface coating
(i) a surface coating material;
(ii) (a) a first enzyme; and
(b) a first substrate, wherein action of the first enzyme on the first substrate provides a second substrate
(iil) a second enzyme, wherein the second enzyme is encapsulated in a silicate, and wherein said second enzyme generates an anti-foulant compound when acting on said second substrate.
